(19) United States
(12) Patent Application Publication
(10) Pub. No.: US 2025/0082406 A1
(43) Pub. Date: Mar. 13, 2025

(54) METHOD FOR SURGICAL PLANNING

(71) Applicant: Hoth Intelligence, Inc., Philadelphia, PA (US)

(72) Inventors: Tyler Alexander, Philadelphia, PA (US); Toryn Farr, Philadelphia, PA (US); Andrew Conklin, Philadelphia, PA (US); Jonathan Cohen, Philadelphia, PA (US); Remi Prince, Philadelphia, PA (US)

(73) Assignee: Hoth Intelligence, Inc., Philadelphia, PA (US)

(21) Appl. No.: 18/882,411

(22) Filed: Sep. 11, 2024

Related U.S. Application Data
(60) Provisional application No. 63/537,741, filed on Sep. 11, 2023.

(51) Int. Cl. A61B 34/10

(52) U.S. Cl. CPC A61B 34/10; A61B 2034/105; A61B 2034/107

(57) ABSTRACT

A surgical assistance method can include: generating a 3D representation of a set of anatomical structures, registering the 3D representation with a subject, displaying an overlay of the 3D representation, determining a trajectory, determining surgical guides, determining observation parameters, and/or facilitating the surgical procedure. The method functions to generate a virtual cut displayed on a 3D representation of a subject to facilitate provider planning and/or performance of a surgical incision.

### CROSS-REFERENCE TO RELATED APPLICATIONS

This application claims the benefit of U.S. Provisional Application No. 63/537,741 filed 11 Sep. 2023, which is incorporated in its entirety by this reference.

### TECHNICAL FIELD

This invention relates generally to the surgical assistance field, and more specifically to a new and useful method for determining a cut and/or trajectory in the surgical assistance field.

### BRIEF DESCRIPTION OF THE FIGURES

FIG. 1 is a schematic representation of a variant of information flow in the method.

FIG. 2 is a schematic representation of a variant of the method.

FIG. 3 is an illustrative example of a variant of a cut guide.

FIGS. 4A-4C are illustrative examples of a variant of displaying volumetric path intersections.

FIG. 5 is an illustrative example of a variant of a cut guide.

FIGS. 6A-6D are illustrative examples of a variant of determining a set of cut guides.

FIG. 7 is an illustrative example of a variant of a cut guide.

FIG. 8 is an illustrative example of a variant of a volumetric path and a volumetric view section.

FIG. 9 is an illustrative example of a variant of facilitating the procedure.

FIG. 10 is an illustrative example of a variant of a virtual sensor.

FIG. 11 is a schematic representation of a variant of trajectory determination.

FIG. 12 is an illustrative example of a variant of registration.

FIG. 13 is a schematic representation of a variant of determining a trajectory.

FIG. 14 is a schematic representation of a variant of determining surgical guides.

FIG. 15 is a schematic representation of a variant of determining a trajectory based on the target.

FIG. 16 is an illustrative example of a variant of a coordinate system.

FIG. 17 is an illustrative example of a variant of a virtual 2D screen.

### DETAILED DESCRIPTION

The following description of the embodiments of the invention is not intended to limit the invention to these embodiments, but rather to enable any person skilled in the art to make and use this invention.

### 1. Overview

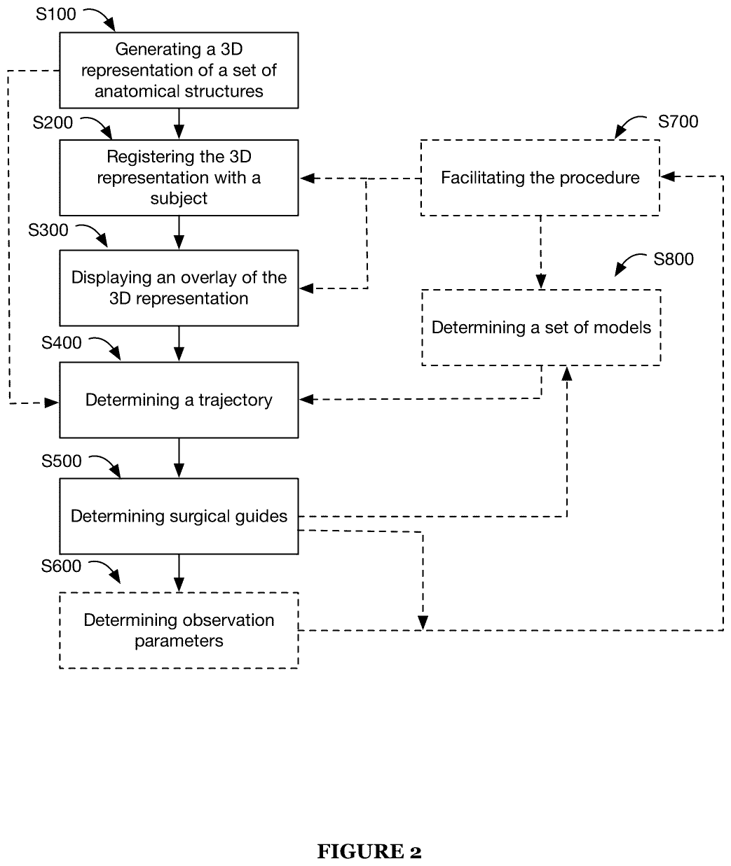

As shown in FIG. 2, a method for determining a cut trajectory can include: generating a 3D representation of a set of anatomical structures S100, registering the 3D representation with a subject S200, displaying an overlay of the 3D representation S300, determining a trajectory S400, determining surgical guides S500, determining observation parameters S600, facilitating the procedure S700, and/or determining a set of models S800. The method functions to generate surgical guides displayed on a 3D representation of a subject to facilitate surgical planning and/or surgical performance. In examples, the method can: project an internal target structure onto the patient's exterior (e.g., for tumor delineation, for resection planning); visualize the internal structures surrounding the resection path; and/or provide other functionalities.

In an illustrative example, a 3D representation of a subject's head can be generated from a set of CT scans, and structures in the 3D representation 100 can be labeled manually and/or automatically. Before cutting into the subject's head to remove a target structure (e.g., a tumor), a set of surgical guides (e.g., a volumetric path 30, a volumetric view section 40, a set of cut guides 50, etc.) can be generated based on the 3D representation 100 virtually overlaid on the subject's body in a display 200 of a 3D headset worn by a surgical provider. A 3D point on the target structure can be chosen and a trajectory 20 for a procedure can be chosen (e.g., by moving a pointer over the subject's body, which can be captured by a sensor 400) and added to the 3D representation 100. The pointer can be a tool tip, a feature of a provider structure (e.g. a tool tip, etc.), a marker (e.g., a QR code), a marking drawn on the subject's skin, and/or any other suitable type of pointer. Within the 3D representation 100, a trajectory intersecting both the 3D point on the target structure and a pointer coordinate can be created (e.g., a linear trajectory extending between the 3D point and the target structure). A volumetric path 30 can be generated by projecting the shape of the target 10 along a trajectory 20 to the pointer coordinate; projecting the shape of a surgical tool along the trajectory to the target; and/or otherwise generated.

Additionally or alternatively, a set of cut guides 50 can be automatically generated for external structures (e.g., surface of a subject's skin, etc.) and/or internal structures (surfaces of internal structures, etc.). Cut guides 50 can be generated by capturing the intersection of the volumetric path 30 and virtual structures 60 within the 3D representation of the subject's body (e.g., head). For example, a cut guide 50 can include an orthographic projection of the target structure onto a patient's exterior surface (e.g., skin) along the trajectory. In variants, this can assist with surgical delineation during surgical planning to ensure that the surgeon has adequate access to the target structure, assist with trajectory determination, and/or be otherwise used.

Additionally or alternatively, a volumetric view section 40 larger than the volumetric path 30 can be generated about the trajectory 20 (e.g., a conical view section, a cylindrical view section, etc.), which can define a region around the volumetric path 30 to display adjacent structures (e.g., critical structures, a predetermined class of structures, all structures, etc.). In a specific example, when the adjacent structures intersect the volumetric view region, the adjacent structures can be displayed to the provider (e.g., surgeon); and the adjacent structures which intersect the volumetric path 30 can be highlighted. The provider can repeat the process of adjusting trajectory parameters (e.g., pose, etc.) until the provider discovers and chooses a suitable trajectory 20. The chosen trajectory 20 and a subset of internal structures within the head can be displayed in the extended reality headset and aligned to structures on the subject's body from the surgeon's point of view.

During surgery, the surgical guides can affect how virtual structures 60 are displayed to the surgeon. In an illustrative example, during surgery, a surgical tool can be tracked relative to the surgical guides, and the system can send a notification to a provider when a provider structure is near (e.g., within a buffer distance from) or intersects a critical structure. In another illustrative example, as the surgical tool moves around, virtual structures 60 nearby the tool can change their appearance (e.g., changing color, opacity, adding a tag, etc.). For example, structures within a predetermined distance of the tool (e.g., tool tip, entire tool, etc.) can be displayed, while structures further away from the tool can be hidden or displayed less prominently.

However, the method can be otherwise performed.

### 2. Technical Advantages

Variants of the technology can confer one or more advantages over conventional technologies.

First, the method enables the surgeon to plan on a 3D surface (e.g., the curved shape of a skull) in 3D space without referencing a 2D representation, unlike conventional methods, which use a 2D representation of the surgical cut to inform the shape of the 3D surgical cut. The method enables subject-specific and target-specific (e.g., tumor-specific) surgical planning by showing how a planned surgical operation affects the surrounding anatomy.

Second, the method helps a surgeon evaluate and choose the best entry point and/or angle of approach for a cut targeting a specific structure within a subject's body, which may be accessed from multiple different angles. In an example, a surgeon may choose to cut along a trajectory 20 which minimizes interference with vital anatomy or regions, minimizes surgery time, minimizes the size of a cut, minimizes cut distance, minimizes the cut perimeter, minimize interference with implants or surgical devices, minimizes the predicted recovery time, and/or optimizes for any other quantitative or qualitative metric. Helping inform the surgeon's decision can make surgeries safer and more effective.

Third, the method minimizes cognitive load in three ways. First, variants of the method keeps a surgeon's focus on the subject, rather than forcing the surgeon to look back and forth between a subject and a set of screens describing the subject's anatomical structures and/or a planned cut path (e.g., a volumetric path 30, etc.). In a variant, the method can accomplish this by displaying information in an extended reality (XR) headset, wherein the surgery is performed primarily based on information displaying within the headset. This process also saves the surgeon time, allowing the surgeon to finish the procedure more quickly and/or take more time on each element of the procedure. Second, by generating a 3D cut guide 50 rather than a 2D cut, the surgeon does not need to mentally project the 2D cut onto the 3D subject—the projection is already performed for the surgeon. This process allows the surgeon to focus more on other important aspects of the procedure. Third, the method eliminates distractions by only displaying relevant structures for surgery (e.g., structures intersecting a volumetric view section 40, etc.).

Fourth, the method enables complex surgical assistance during a procedure. In examples, the system can dynamically respond to a position of a tool (e.g., observed by a camera) by warning the provider when the tool is intersecting or near a critical feature and/or by depicting a cut guide in relation to the tool. Additionally, the method enables the planning of specific cuts on both internal and external structures of the subject in order to minimize unnecessary extraction of material (e.g., brain sections, etc.).

However, further advantages can be provided by the method disclosed herein.

### 3. System

The method can be performed with a 3D representation 100 including virtual structures 60 which represent physical structures of a subject (e.g., a target 10, etc.), a display 200, measurements 300, sensors 400, a trajectory determination model 600, an alignment module 700, a reconstruction module 800, surgical guides (e.g., a volumetric path 30, volumetric view section 40, set of cut guides 50, etc.), and/or any other suitable system components. The system functions to facilitate trajectory 20 determination and/or a surgical procedure.

The display 200 functions to show information about a procedure to a medical provider. The information about a procedure can include measurements 300, the 3D representation 100 and/or virtual structures 60 thereof, other virtual structures 60 (e.g., representing provider structures, etc.), surgical guides, a trajectory 20, notifications, and/or any other suitable information about a procedure. A display 200 can be within a wearable device worn by the provider or can be separate from the provider. The display 200 can include a single window or multiple windows (e.g., displaying picture-in-picture information, etc.). The display 200 preferably shows information in multiple layers (e.g., a subject external anatomy layer, a subject internal anatomy layer, a target layer, a surgical guide layer, etc.) but can alternatively show information in one layer (e.g., a 2D layer, 3D layer, etc.).

The display 200 can be a display of a variety of types. In a first variant, the display 200 can be a virtual reality (VR) display. In an example of this variant, the measurement 300 and the surgical guides are depicted on a single non-transparent screen (e.g., including or not including a lens). In this variant, the display 200 can depict measurements of the adjacent environment sampled by an onboard sensor 400 (e.g., a camera, etc.) in real-time or near-real time. Alternatively, the display 200 can depict only the subject and/or the 3D representation 100 in addition to surgical guides. In a second variant, the display 200 can be an augmented reality (AR) display. In this variant, the display 200 can include a transparent or semi-transparent surface (e.g., a lens and/or feature thereof, etc.) on which information can be projected (e.g., aligned with the subject as viewed through the transparent surface, etc.). In this variant, virtual structures 60, surgical guides, and/or other information are registered with the subject and projected onto the transparent surface. In this variant, information can be projected onto a transparent surface visible to both provider eyes, a transparent surface visible to one provider eye, two transparent surfaces each visible to a provider eye, and/or any other suitable arrangement of transparent surfaces. In a third variant, the display 200 can be a standalone monitor (e.g., proximal the subject, etc.). In a fourth variant, the display 200 can be a portable device (e.g., a smartphone, etc.). In this variant, the display 200 can show information from a fixed viewpoint, from a viewpoint of a sensor 400 of the portable device (for example, a provider can hold the portable device between themself and the subject), from a virtual viewpoint within the subject (e.g., a pose of a virtual sensor 1000), and/or from any other suitable viewpoint. In a fifth variant, the display 200 can be the surface of a subject structure (e.g., the skin of the subject). In this variant, information can be directly projected onto the skin of the subject (e.g., using a projector mounted above the subject, etc.). However, any other suitable type of display 200 can be used.

However, the display 200 can be otherwise configured.

Measurements 300 function to provide information about a subject. Measurements 300 preferably include representations of the subject and can additionally or alternatively include representations of provider structures (e.g., medical tools, a surgeon's hands, etc.), environmental features, markers (e.g., affixed to the subject, etc.), and/or any other suitable information. Subjects are preferably human but can alternatively be animals. Measurements 300 can be sampled during surgical planning, during surgery, and/or at any other suitable time. Measurements 300 can be within a timeseries of measurements 300 (e.g., a video, etc.) but can alternatively not be within a timeseries. In an example, features of a measurement 300 (e.g., keypoints) are determined based on features for prior measurements 300 within the timeseries and optionally motion data (e.g., motion of the sensor 400, etc.). Measurements 300 can include imagery, video, audio, depth information, and/or data in any other suitable modality. In an example, measurements 300 can be RGB images. The system can capture one or more measurements 300 at once (e.g., by different sensors 400 with different poses, etc.).

Measurements 300 can be captured by sensors 400 (e.g., cameras, depth sensor, a LiDAR sensor, etc.). Sensors can be mounted to the display 200 (e.g., an XR headset, etc.), incorporated into a portable device (e.g., a TrueDepth camera on a smartphone, etc.), and/or otherwise located. Sensors can be positioned above a subject, adjacent to a subject, coupled to a provider performing a procedure (e.g., in a headset), and/or otherwise positioned. However, sensors 400 can be otherwise configured.

However, measurements 300 can be otherwise configured.

Physical structures can be structures used to facilitate a procedure, structures on which the procedure is performed, and/or any other suitable structure. Physical structures can represent anatomical structures and/or non-anatomical structures.

Anatomical structures include interior structures (e.g., bones, organs, blood vessels, nerves, fat, abnormal growth, tumor, any body tissues, etc.), exterior structures (e.g., surface structures, head, face, abdomen, extremity, digit, skin, hair, eye, exterior abnormal growth, any region between air—or another medium—and an interior structure, etc.), visible structures, concealed structures (e.g., not depicted in a measurement 300, etc.), partially-concealed structures, and/or any other suitable physical structure of a subject and/or provider. Examples of anatomical structures include the brain, brain regions (e.g., cerebrum, brain hemispheres, white matter, lobes, cerebellum, cortices, brainstem, midbrain, pons, medulla oblongata, diencephalon, thalamus, hypothalamus, limbic system, hippocampus, amygdala, cingulate gyrus, basal ganglia, corpus callosum, ventricles, substantia nigra, fornix, etc.), blood vessels (veins, arteries), nerves, bone (e.g., spine, skull), meninges, cerebrospinal fluid regions, structures within the ear and/or eyes, glands, muscles, lymph nodes, skin, and/or any other suitable anatomical structures. However, anatomical structures can otherwise be characterized.

Non-anatomical structures can include implants, foreign objects, surgical tools, needles, blades, hoses, monitors, deep brain stimulation (DBS) implants, cochlear implants, motor cortex implants, brain-computer interface (BCI) implants, nerve stimulation implants, cranial implants, screws, fiducials, and/or other structures. However, non-anatomical structures can otherwise be characterized.

Physical structures can include subject structures and/or provider structures. Subject structures preferably represent structures associated with the subject, and provider structures preferably represent structures associated with the provider. Examples of provider structures include a hand (e.g., left hand, right hand, etc.), a finger (e.g., index finger, long finger, ring finger, small finger, etc.), a thumb, an arm, a leg, a joint (e.g., of a finger, thumb, arm, leg, etc.), a bone, an eye, a back, a neck, any exterior structure, any interior structure, medical instruments and/or any other feature and/or portion thereof, of a provider and/or a medical instrument. However, provider structures can be otherwise characterized.

Physical structures of a subject (e.g., subject structures) can include target structures (e.g., the target 10) but can additionally or alternatively include surrounding structures. The target structure is preferably a structure to be removed from the subject but can alternatively be a structure to be treated (e.g., injected into, etc.), measured, and/or otherwise affected during the procedure. Examples of target structures include tumors, clots, growths, stones, implants (e.g., expired implants, etc.), bone fragments, foreign objects (e.g., bullets, etc.), cysts, parasites, and/or any other suitable type of physical structure. The target structure preferably has an irregular shape but can alternatively have a regular shape. However, target structures can be otherwise characterized.

Surrounding structures can be non-target structures (e.g., structures which surround the target structure, structures near the target structure, etc.). In an example, surrounding structures and/or regions thereof can optionally be assigned (e.g., flagged) with a set of criticality scores indicating an importance of the surrounding structure and/or region thereof. In this example, sets of criticality scores can be used to calculate an intersection score for a trajectory 20 and/or surgical guide which intersects and/or is close to the corresponding surrounding structures.

Surrounding structures can optionally include a secondary structure (e.g., a destination structure, external structure, etc.). The secondary structure can be used for: virtual model—patient registration and tracking; trajectory determination; and/or otherwise used. The secondary structure can include a point and/or surface which can bound the trajectory 20 at an opposite end as the target 10 (e.g., a second end region, etc.). Examples of secondary structures include the skin of a subject, the skull of a subject, and/or other suitable structures. The trajectory 20 is preferably determined based on the secondary structure, but the secondary structure can alternatively be determined based on the trajectory 20. However, the secondary structure can otherwise be characterized.

However, surrounding structures can otherwise be characterized.

However, physical structures can otherwise be characterized.

Virtual structures 60 function to represent physical structures. Virtual structures 60 can represent any suitable type of structure (e.g., subject structures, provider structures, target structures, surrounding structures, secondary structures, anatomical structures, non-anatomical structures, etc.); alternatively, virtual structures 60 can exclude representations of any of the aforementioned structure types. In an illustrative example, virtual structures 60 can include both internal and external structures, wherein the internal structures are aligned with the external structures (e.g., from 3D representation generation), and the external structures include keypoints (e.g., for alignment with the subject representation). The virtual structures 60 can be referenced to or share a common reference frame (e.g., generated from the same set of subject scans), registered, and/or otherwise aligned. Virtual structures 60 preferably exist within a 3D representation of a subject 100 but can alternatively be separate from the 3D representation 100. Preferably, the 3D representation 100 excludes provider structures, but the 3D representation 100 can alternatively include provider structures. Virtual structures 60 can be overlapping or non-overlapping within the 3D representation 100 (e.g., a “brain” virtual structure 60 and a “frontal lobe” virtual structure 60 can coexist in the 3D representation 100). The 3D representation 100 is preferably a virtual model (e.g., hull, shell, point cloud, etc.), but can additionally or alternatively include a set of labeled positions (e.g., different positions labeled with the structure type) and/or be otherwise represented. The 3D representation 100 can be generated from imagery (e.g., 2D imagery, 3D imagery, scans, etc.), scans 310 (e.g., CT scans, MRI scans, PET scans, ultrasound imagery, etc.), and/or any other suitable information. The 3D representation 100 is preferably generated in S100 but can alternatively be generated at any other suitable time. In variants, the 3D representation 100 can be the “patient-specific image reconstructed model” “3D representation” and/or “3D representation” disclosed in U.S. application Ser. No. 17/719,043, filed 12 Apr. 2022, incorporated herein in its entirety by this reference, or be any other model. However, the 3D representation 100 can be otherwise configured.

Virtual structures 60 can represent external structures (e.g., skin, hair, etc.) and/or internal structures (e.g., brain lobes, organs, etc.), and/or any other suitable structure type. External structures preferably represent physical structures which include features visible and/or accessible from the outside of the subject (e.g., before a surgery; alternatively during a surgery, for example, through a surgical opening; etc.), but can alternatively refer to other suitable structures. Internal structures preferably represent physical structures which include features not visible and/or accessible from the outside of the subject (e.g., are occluded by skin and/or other structures). Virtual structures 60 representing internal structures and virtual structures 60 representing external structures are preferably aligned with each other (e.g., based on a relative position measured in the scans 310 and/or 3D representation 100 generated therefrom, etc.) but can alternatively not be aligned with each other. In an example, the 3D representation 100 can include a set of virtual external structures (e.g., bone, soft tissue, skull, skin, etc.) aligned with a set of virtual internal structures (e.g., target structures, vital organs, critical structures, etc.), both representing the size, shape, and pose of their respective physical analogs.

Pose and/or other characteristics of a physical structure represented by a virtual structure 60 can be inferred based on the states of the virtual structure 60. For example, the physical structure characteristics can be determined based on observed relationships between observed features (e.g., keypoints extracted from the measurement 300) and virtual structure features (e.g., keypoints of the virtual structure 60 corresponding to the physical structure). In a first example, the pose of a physical structure associated with a virtual structure 60 can be determined based on the states of a set of observed features of the physical structure (e.g., where part or all of the physical structure can be visible). In a second example, the pose of the physical structure associated with a virtual structure 60 can be determined based on a set of observed features of other physical structures with a known relative pose relative to the virtual structure 60 associated with the subject structure.

Virtual structures 60 can include keypoints (e.g., for registration). Keypoints preferably correspond to features of external structures which are visible to a sensor 400 external to the subject (e.g., tip of nose, etc.) but can alternatively correspond to other suitable features. Keypoints can correspond to patient anatomy, markers (e.g., a QR code), markings (e.g., drawn on the surface of a patient, etc.), and/or any other suitable feature. In an example, S200 can include using the keypoints of the virtual structures 60 to register the 3D representation 100 with a measurement 300 by matching keypoints of the virtual structures 60 to corresponding keypoints within the measurement 300. In examples, keypoints can indicate the location of: a right ear, a left ear, a mouth, a nose, a forehead (e.g., center of the forehead), a center of mass, an abnormal growth, a surgical incision point (e.g., Kocher points), and/or any other point of interest. Preferably, keypoints are represented by a set of 3D coordinates, but can alternatively be represented by a set of 2D coordinates, and/or otherwise represented. Keypoints can be identified from a measurement 300 using any keypoint detection model (e.g., object detector, classifier, segmentation model, etc.), and/or by any other suitable system component. Keypoints can be identified for a virtual structure 60 using a labeling model 900 and/or any other suitable model or system component. In variants, the labeling model 900 can be trained to label virtual structures 60 with keypoints, can be trained to label scans 310, and/or can be otherwise trained. In a where the labeling model 900 labels scans 310, resultant labels (e.g., tags) and/or keypoints can be transferred to corresponding regions of virtual structures 60 determined from the scans 310. However, keypoints can be otherwise characterized.

Virtual structures 60 and/or regions thereof can optionally be tagged. In variants, tags can be a criticality score, a penalty score (e.g., penalty for intersecting the structure), a designation of a “no go” zone, a label indicating structure type (e.g., organ type, brain region, etc.), a “cut here” instruction, and/or any other suitable type of tag. Virtual structures 60 can be manually or automatically tagged (e.g., based on structure type, etc.). However, virtual structures 60 can otherwise be tagged or not tagged.

However, virtual structures 60 can be otherwise determined.

The trajectory 20 functions to represent a path between a target 10 and a secondary structure (e.g., a tumor extraction path, etc.). The trajectory can be defined by trajectory parameters specifying a first end region (e.g., first endpoint), second end region (e.g., second endpoint), length, angle (e.g., relative to the target 10, relative to a first end region, etc.), shape (e.g., a linear path, curved path, etc.), dimensions (e.g., length, thickness, slant, etc.), and/or any other suitable attribute of a trajectory 20. The trajectory 20 preferably has a first end region and a second end region (e.g., a first and second endpoint) but can alternatively have any other suitable number of end regions. The first end region is preferably on the target 10 (e.g., at a target point, on a target structure, etc.) but can alternatively be off the target 10. Examples of first end regions include a target structure, a center of mass of the target 10, center of volume of the target 10, a proximal point to the second end region, a distal point from the second end region, a manually-selected point, a point on a surface of the target 10, a surface of the target 10, and/or another suitable first end region. The second end region is preferably on the secondary structure but can alternatively be off the secondary structure. Examples of second end regions include a point on a subject exterior, a secondary structure (e.g., and/or a point or surface thereon), a tool tip (e.g., a pointer coordinate, etc.), a fingertip (e.g., of a provider, etc.), a provider-defined region, and/or any other suitable second end region. The first and second end regions can be defined within a coordinate system of the subject, a coordinate system of the 3D representation 100, a coordinate system of a provider structure, a global coordinate system, and/or any other suitable coordinate system. In an illustrative example, the trajectory 20 can be a straight line in 3D space between the first end region at the center of a tumor and second end region on the surface of a skull.

The trajectory 20 can be linear, curved, regular, irregular, and/or any other suitable shape. In an example, the trajectory 20 can be a line between a first end region and a second end region. In a second example, the trajectory 20 can have a non-linear shape which can be determined by constraints associated with a surgical tool (e.g., dimensions, shape of the tool, etc.). The trajectory 20 can be a manually-drawn path and/or can be automatically determined. The trajectory 20 is preferably determined after the first end region is determined and concurrently with the second end region being determined, but the trajectory 20 can alternatively be determined at any other suitable time. The trajectory 20 can be determined by a provider, by a trajectory determination model 600, and/or any other suitable system component. However, the trajectory 20 can be otherwise characterized.

The optional trajectory determination model 600 functions to determine trajectory parameters for a surgical procedure. The trajectory determination model 600 can determine a trajectory 20 based on a target 10 (e.g., a target structure, etc.); and optionally a secondary structure (e.g., a layer of skin), surrounding structures and tags thereof, surgical tool constraints, and/or any other suitable information. The trajectory determination model 600 can accept as inputs a set of virtual structures 60, a first end region, a second end region, information about a subject's body, information about a target structure, information about surrounding structures (e.g., and tags thereof, etc.), information about available surgical tools and methods, time constraints, procedure type, target parameters, secondary structure parameters, trajectory constraints, and/or any other suitable of information. The trajectory determination model 600 can output trajectory parameters (angle, length, shape, etc.), a first end region, a second end region, modified non-target structures, data, cut parameters, and/or any other suitable information. The trajectory determination model 600 is preferably trained and/or refined in S800. Target values (for training) can include prior determined trajectories 20 (e.g., trajectories 20 selected during previous surgeries, trajectories 20 determined in previous iterations of the S400 step, etc.) and/or trajectories 20 determined through any other method. The trajectory determination model 600 can be a ML model (e.g., a DNN, etc.) or any other suitable type of algorithm. The trajectory determination model 600 can be trained using supervised learning, unsupervised learning, a mix of training methods, and/or any other suitable methods. However, the trajectory determination model 600 can be otherwise configured.

Surgical guides function to provide additional information relating to the surgery to a provider. Surgical guides are preferably aligned with the 3D representation 100, but can alternatively be aligned with the subject represented in the measurement 300 directly (e.g., via keypoints) and/or be aligned with any other suitable environmental features. In an example, a surgical guides can be aligned with the trajectory 20. In a second example, a surgical guide can be aligned with a virtual structure 60 representing the target 10. In a third example, a surgical guide can be aligned with a virtual structure 60 representing the surface of the subject (e.g., and/or keypoints thereof, etc.). In a fourth example, a surgical guide can be aligned with a surrounding structure (e.g., an observed surrounding structure closest to the target 10, etc.). However, the surgical guides can be otherwise aligned. Surgical guides can be determined within a virtual space (e.g., defined by the 3D representation 100), within a measurement space (e.g., an image space), and/or physical space. Surgical guides can be displayed within a 3D reconstruction layer, over the 3D reconstruction layer, and/or otherwise displayed.

Surgical guides can include one or more of: a volumetric path 30, a volumetric view section 40, a set of cut guides 50, and/or any other suitable type of surgical guide.

A volumetric path 30 functions to virtually represent a 3D volume through which a target 10 (and/or portions thereof) travel. The volumetric path 30 is preferably determined in S510 but can alternatively be determined at another suitable time. The volumetric path 30 is preferably a 3D shape connecting the target 10 and a second end region (e.g., preferably the same second end region as the trajectory 20 and/or volumetric view section 40, alternatively a different second end region, etc.) but can alternatively have another shape and/or orientation. The volumetric path can extend along the trajectory, extend adjacent the trajectory, extend independently from the trajectory, and/or be otherwise related to the trajectory. In an example, the volumetric path 30 can encompass both the target 10 and the second end region. In a second example, the volumetric path 30 can extend beyond the target 10, the second end region, and/or the secondary structure. In examples, the volumetric path 30 can be a resection path.

The path profile of the volumetric path 30 (e.g., the cross-section of the volumetric path 30 normal to the trajectory 20) is preferably irregular (e.g., tracing the irregular boundary of the target 10 and/or a portion thereof, etc.) but can alternatively be regular. The path profile can preferably be smaller than the tumor (e.g., the shape/size of a tumor chunk; the shape/size of a surgical tool), be the same size as the tumor or larger than the tumor, be manually defined, be automatically defined (e.g., based on the diameter of the surgical tool, based on the size of the target structure, etc.), and/or be otherwise defined.

In a first embodiment, the path profile can have a shape based on the target 10. In a first variant, the path profile can be a cross-section of the target 10. In this variant, the cross section of the target 10 can be: the cross section orthogonal to the trajectory 20 at the first end region, a minimum diameter cross section, a maximum diameter cross section, minimum area cross section, maximum area cross section, or another suitable cross section. In a second variant, the path profile can be a projection of the target 10 (e.g., a silhouette in the direction of the trajectory 20). In a third variant, the path profile can approximate the target shape (e.g., a circle, an oval, a triangle, a polygon, a minimum convex path around a cross section or silhouette, etc.). In a fourth variant, the path profile can be a shape drawn manually by the surgeon (e.g., in 3D space, on the secondary surface, etc.). In a fifth variant, the path profile shape can be based on shapes in any of the aforementioned variants (e.g., transformed by adding an inner margin to make the path profile smaller, etc.) However, the path profile can be otherwise characterized.

In a second embodiment, the path profile can have a shape based on a surgical tool used in the procedure (e.g., suction tip, forceps, needle, blades, clips, etc.). In such embodiments, the path profile can be a cross-section of a shape of a surgical tool, a silhouette of a surgical tool and/or portion thereof, and/or another suitable shape.

In a third embodiment, the path profile can have a shape based on the largest region of a target 10 planned for extraction at one time (e.g., in a variant where the tumor is extracted in chunks, etc.).

However, the path profile can be otherwise defined.

The volumetric path 30 is preferably continuous but can alternatively be discontinuous. The volumetric path 30 preferably has a constant path profile along the trajectory 20 but can alternatively have a varying path profile along the trajectory 20. In an illustrative example, the volumetric path 30 can be a region which represents the access path between the skin of a subject and the target 10 (e.g., a tumor, etc.). In this illustrative example, the volumetric path 30 can be generated by volumetrically projecting the path profile (e.g., a silhouette of the target 10 along the trajectory 20) along the trajectory 20 up to the surface of the skin. However, the shape of the volumetric path 30 can be otherwise characterized.

However, the volumetric path 30 can be otherwise characterized.

The volumetric view section 40 functions to virtually represent a 3D volume which facilitates viewing of the target 10 and optionally adjacent structures during procedure planning and/or during the procedure. The volumetric view section 40 is preferably determined in S520 but can alternatively be determined at another time. The volumetric view section 40 is preferably larger than the volumetric path 30 but can alternatively be the same size or smaller than the volumetric path 30. Alternatively, the volumetric view section 40 can be the volumetric path 30. The volumetric view section 40 is preferably a 3D shape connecting the target 10 and the second end region (e.g., preferably the same second end region as the trajectory 20 and/or volumetric path 30; alternatively a different second end region, etc.) and/or secondary structure but can alternatively have another suitable shape. The volumetric view section 40 can optionally extend beyond the target 10, second end region, and/or secondary structure. The shape of the volumetric view section 40 can be determined based on the volumetric path 30, target position, target shape, target size, trajectory 20, an outer margin value, a volumetric view angle (e.g., frustum apex angle, half-apex angle, etc.), and/or another suitable shape. The volumetric view section 40 is preferably a frustum (e.g., tapered) shape (e.g., with a small base at the target 10 and a large base at the second end region) but can alternatively be another suitable shape. In an example, the volumetric view section 40 can be centered around the trajectory 20, around a boundary surrounding the trajectory 20, and/or another feature. The cross-sectional shape (e.g., view section profile) of the volumetric view section can be circular, obolid, polygonal, the shape of the volumetric path 30, the shape of the target 10, the shape of a cut, a provider-defined shape, and/or any other suitable shape. The view section parameters (e.g., aperture angle, the base size(s), the pose of the view section centerline, etc.) can be automatically determined (e.g., based on the target structure size, the volumetric path, etc.), manually determined, and/or otherwise determined. The view section profile is preferably irregular but can alternatively be regular. The view section profile size can be the size of the target cross section, the size of a cut, and/or another suitable size. The view section profile size can be constant, can monotonically increase from the target 10 to the secondary structure and/or second end region, and/or can otherwise change. The volumetric view section preferably follows the shape of the volumetric path 30 but can alternatively follow a different shape.

In an illustrative example, the volumetric view section 40 can be a region which facilitates filtering of virtual structures 60 within the 3D representation of the subject, such that only virtual structures 60 near to the volumetric path 30 are depicted. In this illustrative example, the volumetric view section 40 can be generated by adding a volumetric margin (e.g., an outer margin in the radial direction relative to the trajectory 20, etc.) to the volumetric path 30.

However, the volumetric view section 40 can be otherwise configured.

The set of cut guides 50 function to virtually represent, guide, and/or assist in planning a surgical cut. The set of cut guides 50 are preferably determined in S530 but can alternatively be determined at another suitable time. The set of cut guides 50 can be 2D or 3D (e.g., through the thickness of a surrounding structure, along a 3D surface of a surrounding structure, etc.). In a first variant, a cut guide 50 can be two dimensional. In a second variant, a cut guide 50 can be a 3D path constrained to a surface of a structure (e.g., skin, an internal structure, etc.). In an example, the cut guide 50 can delineate the tumor margin, which can be used by the surgeon to plan the cut (e.g., to remove all or most of the tumor). In further variants, a representation of the tumor can be virtually slid along the trajectory to determine the tumor margin in the depth direction. In a third variant, a cut guide 50 can be a slice of a 3D structure. However, cut guides 50 can be otherwise shaped. Cut guides 50 can have different observation parameters information relating to the surgery (e.g., different colors for cut guides 50 for different targets 10, different colors for cut guides 50 along different surrounding structures, different colors for different levels of criticality of an intersected structure, etc.). The cut guide 50 can be determined using a virtual representation of a secondary structure (e.g., a virtual structure 60, a video, an image, and/or another virtual representation), the actual surface of the structure (e.g., the skin of the subject, etc.), and/or any other real or represented structure. The cut guide 50 can be determined by taking the intersection of the volumetric path 30 (and/or boundary thereof) with a virtual structure 60 within the 3D representation 100 but can alternatively be determined. The cut guide 50 can be depicted on a subject representation, on a representation of a virtual structure 60, can be projected onto the skin of a subject (e.g., using a projector, etc.), and/or can otherwise be depicted. The cut guide 50 can be fixed or dynamic (e.g., modified during the procedure; for example, removed when the cut guide 50 can be executed by a surgical tool, etc.). However, the set of cut guides 50 can otherwise be configured.

However, surgical guides can otherwise be characterized.

However, the system can otherwise be configured.

### 4. Method

The method can include: generating a 3D representation of a set of anatomical structures S100, registering the 3D representation with a subject S200, displaying an overlay of the 3D representation S300, determining a trajectory S400, determining surgical guides S500, determining observation parameters S600, facilitating the procedure S700, and/or determining a set of models S800. The method and/or portions thereof can be performed using a single processing system 500 or multiple processing systems 500.

All or portions of the method can be performed one or more times for a given subject; repeated for different subjects; and/or otherwise performed. In an example, portions of the method (e.g., S300, S700, etc.) can be repeated iteratively during a procedure planning process and/or during a procedure based on measurements 300 of a changing subject.

Generating a 3D representation of a set of anatomical structures S100 functions to create a set of virtual structures 60 representing a subject's body. S100 can be performed immediately before a surgical procedure (e.g., using a 3D representation 100 generated based on scans captured of the unconscious subject before surgery), in a different session, and/or at any other suitable time. S100 can be performed within 1 day before surgery, within 2 hours of surgery, within 1 hour of surgery, within 30 minutes of surgery, within 10 minutes of surgery, within 5 minutes of surgery, within an open or closed time period bounded by the aforementioned values, and/or at any other suitable time relative to surgery. However, S100 can be performed at any other suitable time.

S100 can be performed by the same or different processing system(s) the processing system(s) performing S200-S800.

A 3D representation 100 (and/or virtual structures 60 within the 3D representation 100) can include keypoints (e.g., those used in S200), structures, and any other elements.

The 3D representation 100 is preferably generated from a set of scans 310 of a subject, but can additionally or alternatively be generated from a set of measurements and/or any other suitable information. The 3D representation 100 preferably does not include the set of scans 310 and/or artifacts thereof but can alternatively include the set of scans 310, a subset of the set of scans 310, artifacts of the set of scans 310, and/or other data from the set of scans 310.

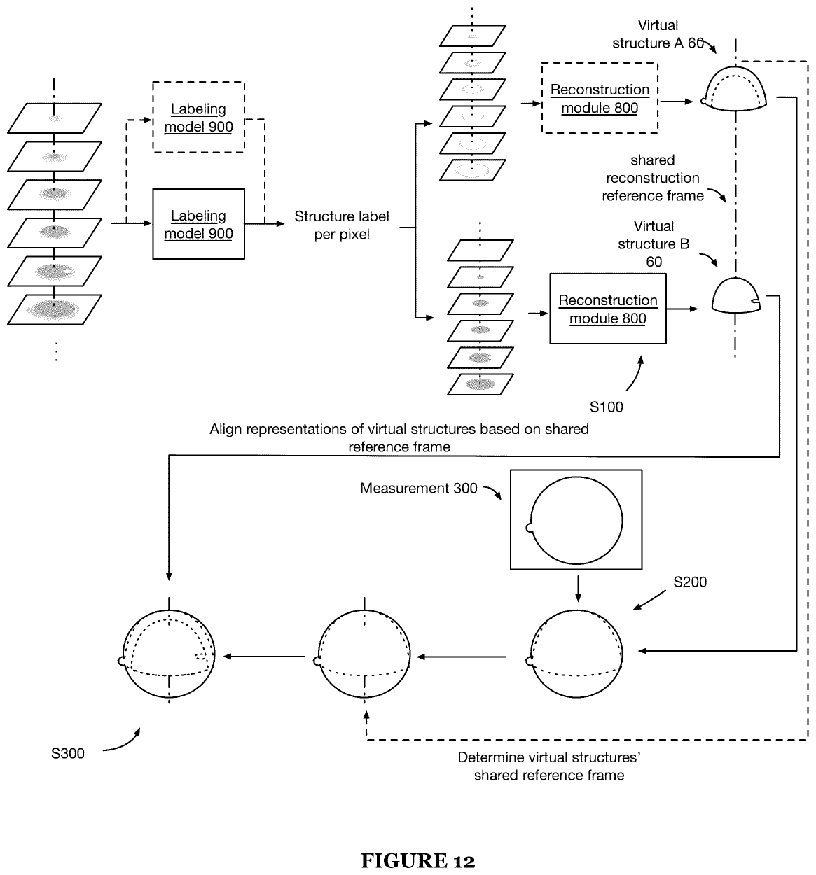

In a first variant, a 3D representation 100 can be generated using a reconstruction module 800 based on scans 310 of a subject (e.g., example shown in FIG. 12). The scans 310 can include a set of 2D and/or 3D scans of a subject. Examples of scans 310 include CT scans, x-rays, MRIs, ultrasound scans, PET scans, SPECT scans, DEXA scans, Fluoroscopy scans, mammography scans, endoscopy scans, and/or any other suitable type of scan. The scans 310 can be labeled or unlabeled with anatomical structures. In an example, labeled scan segments can be labeled with the same labels used for the virtual reconstruction of the corresponding anatomical structure.

In a second variant, the 3D representation 100 can be retrieved from storage, wherein the 3D representation 100 was determined using a previous interpretation of a set of scans 310 of the subject.

In a third variant, structures can be manually or automatically generated and added to the 3D representation 100. In an example of this variant, virtual structures 60 can be amended based on measurements 300 captured during a procedure (e.g., a position of a target structure can be changed during extraction, etc.).

In a fourth variant, the 3D representation 100 can be generated as described in U.S. patent application Ser. No. 17/719,043 filed on 12 Apr. 2022, incorporated herein in its entirety by this reference.

In a fifth variant, the 3D representation 100 can be a generic 3D representation 100 (e.g., not specific to the subject).

However, S100 can be otherwise performed.

Registering the 3D representation with a subject S200 functions to align the 3D representation 100 and/or virtual structures 60 thereof with a subject representation. S200 can optionally additionally or alternatively include aligning virtual structures 60 separate from the 3D representation with the subject representation. S200 is preferably performed after S100 but can additionally or alternatively be performed at any other suitable time. S200 is preferably performed once per subject but can additionally or alternatively be iteratively performed (e.g., periodically, when a new subject representation is received, when a sensor 400 capturing subject representations moves, when a threshold number of alignment keypoints are no longer detected, when another condition occurs, iteratively performed during procedure planning and/or during the procedure, etc.), performed multiple times, or not performed at all (e.g., when the 3D representation 100 can be generated from a synthetic subject representation). In an example, the 3D representation 100 can be re-registered for a new subject representation (e.g., in a measurement 300 of the subject captured from a new angle, in a measurement 300 of a moved subject, etc.). In another example, once the registration between the 3D representation 100 and the subject representation is determined, the registration can be used to maintain the alignment between the 3D representation 100 and the patient as the sensor (e.g., XR headset) moves relative to the patient.

S200 is preferably performed by an alignment module 700 (e.g., running on the processing system 500) but can additionally or alternatively be performed by another suitable system component.

The alignment module 700 can leverage: point set registration (e.g., a point matching algorithm, iterative closest point, such as ICP, N-ICP, etc.; robust point matching; etc.), which registers matching keypoints of virtual structures 60 of the 3D representation 100 (e.g., an exterior structure representation) and keypoints of a subject representation; a spatial mapping algorithm; mesh alignment methods; correspondence-based registration; and/or any other suitable algorithm.

In variants where headset is used, S200 can include using kinematic information for the sensor 400 (e.g., image-based motion tracking, IMU measurements, etc.), depth measurements, visual measurements, IR measurements, and/or other measurements. In such variants, features and/or structures and/or motion thereof can be tracked over time (e.g., to use as priors for a next iteration of S200, for next feature detection, etc.).

The 3D representation 100 and/or virtual structures 60 thereof can be aligned with the physical subject, a subject representation, and/or otherwise aligned with the subject. The subject representation can include: a measurement 300 of a subject (e.g., image, video, depth measurement, etc.), a projection of the subject (e.g., onto a transparent surface or lens of an augmented reality device, etc.), a secondary model of the subject (e.g., of the subject exterior), a proxy object (e.g., for AR displays; alternatively referred to as a “ghost object”, etc.), and/or any other representation thereof. The subject representation can be or be determined from measurements 300 of the subject (e.g., sampled from a headset position, from a projector's position, from any other suitable perspective, etc.); be manually determined; be virtually determined (e.g., be a synthetic subject); and/or be otherwise determined. S200 preferably uses a set of keypoints observed in the subject representation (e.g., a measurement 300 of the subject). Keypoints can be unique visual features (e.g., appearing in an image space, in the depth space, etc.) and/or can additionally or alternatively be unique geometric features (e.g., appearing in a geometric space). Keypoints can alternatively referred to herein as “alignment features”.

In a first variant, S200 includes: identifying keypoints within the virtual structures 60 (e.g., 3D representation keypoints) and the subject representation; matching at least a subset of the keypoints between the virtual structures 60 (e.g., 3D representation keypoints) and a subject representation; and aligning the 3D representation 100 with the subject representation based on matching keypoints (e.g., matching keypoints visible in the subject representation). In this variant, keypoints are preferably globally or locally unique, but can alternatively be nonunique. The keypoints can be a predetermined set of keypoints (e.g., nose tip, inner eye, outer eye, lips, earlobe, armpit, right pointer finger, etc.), or be an undefined set of keypoints (e.g., unique geometric features identified in the virtual structures 60 of the 3D representation 100 and the subject representation). In this variant, S200 can use point matching, iterative closest point (ICP, N-ICP; robust point matching, spatial mapping, mesh alignment methods, correspondence-based registration, and/or any other suitable registration method. In an example, positions of virtual structures 60 within the 3D representation 100 can be estimated based on their relative to features of virtual structures 60 corresponding to keypoints, virtual structures 60 corresponding to keypoints, and/or any other data corresponding to keypoints. Positions of the virtual structures 60 can be determined in a coordinate system defined by the 3D representation 100, in a real-world coordinate system, and/or any other suitable coordinate system. However, the virtual structures 60 of the 3D representation 100 and subject representation can be otherwise aligned using keypoints.

In a second variant, the virtual structures 60 can be manually registered with a subject or subject representation. In this variant, a provider (e.g., a surgeon, practitioner, etc.) manually selects matching points and/or regions of virtual structures 60 of the reconstructed 3D representation 100 and subject representation, and the virtual structures 60 and subject representation are aligned based on matching points and/or regions.

In a third variant, the virtual structures 60 and subject representation can be aligned as described in U.S. patent application Ser. No. 17/719,043 filed on 12 Apr. 2022 incorporated herein in its entirety.

In a fourth variant, the virtual structures 60 can be aligned with the subject representation using a set of known markers (e.g., RF markers, QR codes, etc.), wherein the same known markers can be depicted in both the subject representation and the first set of subject measurements 300 and/or the 3D representation 100. However, the 3D representation can be otherwise aligned.

In a specific example, a measurement 300 of the subject can be sampled and aligned with a 3D representation 100 using keypoints of an exterior structure (e.g., using ICP or another suitable model-matching algorithm). In this specific example, virtual structures 60 can be projected onto the measurement 300 (and/or subject themselves) based on the alignment. A display 200 within a headset can depict an overlay of one or more of the 3D representation 100 over the measurement 300. One or more of the keypoints and/or another suitable point can be tracked (e.g., using SLAM) and used to keep the overlay aligned with the subject (e.g., as the wearer of the headset moves, as the subject moves, etc.). Optionally, tools can be registered and tracked by the headset so that, as the physician inserts a tool or other material inside the subject, a representation of the tool can be depicted over the measurement 300.

Alternatively, S200 can be performed using markers appearing in the scans 310 and the patient measurement (e.g., real-time measurement), markers appearing in the 3D representation and the patient measurement, and/or otherwise determined. However, any other method or combination of methods of registration can be used.

However, S200 can be otherwise performed.

Displaying an overlay of the 3D representation S300 functions to display the 3D representation 100 and/or virtual structures 60 thereof over the subject representation (e.g., image of the subject, subject as seen through lenses, etc.) on a display 200. S300 can be performed as a part of S700 but can additionally or alternatively be performed separately from S700. S300 can preferably be performed after S200, but can additionally or alternatively be performed during and/or after S400, S500, S600, or S700. However, S300 can be performed at any other suitable time. S300 can preferably be continuously performed throughout procedure planning and operation but can additionally or alternatively be intermittently performed throughout procedure planning and/or performance. In an example, S300 can be performed responsive to S200. S300 can use the registration determined in S200 or can use any other alignment. Alternatively, S300 step can occur without aligning the 3D representation 100 with a subject representation.

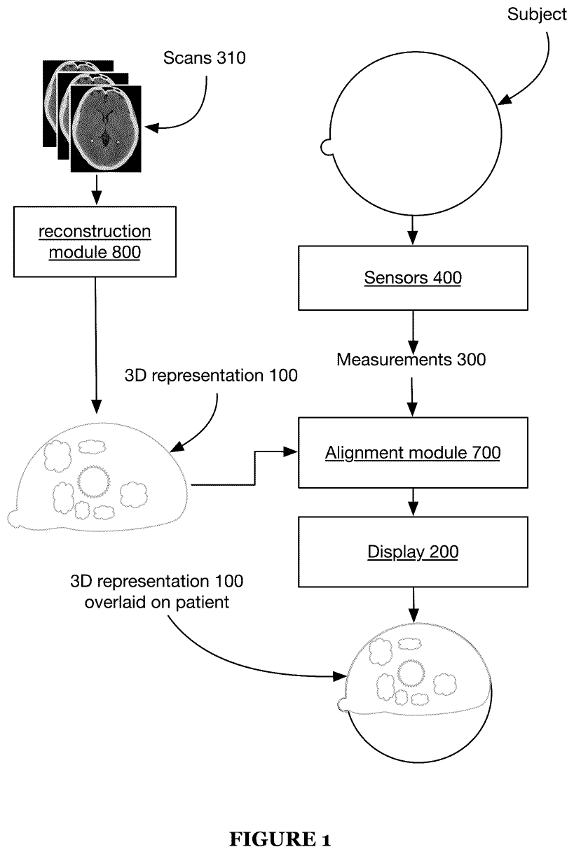

In a first variant, the 3D representation 100 and/or virtual structures 60 thereof can be overlaid over a measurement 300 (e.g., an image or video) of the physical subject using an AR/XR/VR headset (e.g., example shown in FIG. 1), screen, or other display medium. In a first example, the locations of the visible keypoints on the physical subject can be identified and monitored, and the pose (e.g., position and/or orientation) of the 3D representation 100 and/or virtual structures 60 thereof can be adjusted to match the identified keypoint locations on the subject. In a second example, the 3D representation 100 and/or virtual structures 60 thereof can be shown in a window depicting a representation of the physical subject (e.g., video, image, keypoints, etc.).

In a second variant, the 3D representation 100 and/or virtual structures 60 thereof can be projected onto the physical subject (e.g., using a light projector).

In a third variant, S300 can be performed as described in U.S. patent application Ser. No. 17/719,043 filed on 12 Apr. 2022, incorporated herein in its entirety.

In variants, multiple views of the 3D representation 100 and/or virtual structures 60 thereof (and/or trajectory 20, surgical guides, etc.) can be displayed (e.g., in a single display 200 or in multiple displays 200). The primary view is preferably from the perspective of the provider (e.g., surgeon), headset, or real-world sensor 400, but can alternatively be from another perspective. Secondary views can be from virtual sensors 1000, secondary real-world sensors 400, and/or other perspectives. Secondary views preferably have a different perspective from the primary views but can additionally or alternatively have similar perspectives.

In variants, any other suitable type of display 200 can be used.

However, any other method or combination of methods for displaying a 3D representation 100 can be used.

The appearance of virtual structures 60 of the 3D representation 100 (and/or other virtual structures 60) can be based on a trajectory 20 (e.g., determined in S400), surgical guides (e.g., determined in S500), observation parameters (e.g., determined in S600), and/or other information. However, the appearance of virtual structure can alternatively be independent of observation parameters, or otherwise determined.

However, S300 can be otherwise performed.

Determining a trajectory S400 functions to determine a 3D access trajectory to an internal structure for a surgical procedure. In an illustrative example, the trajectory 20 represents the access path from the surface of a head to a brain tumor and/or the tumor removal path (e.g., from the in situ tumor location to the head surface).

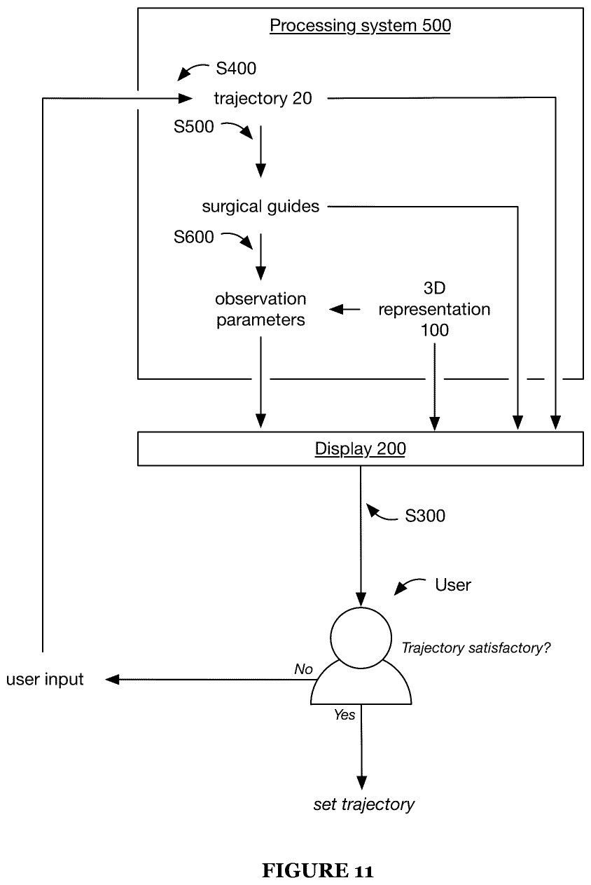

Determining a trajectory S400 can be performed once or repeated (e.g., iteratively repeated with S500; repeated to generate multiple candidate trajectories, example shown in FIG. 11; etc.) and/or performed in any other suitable manner. S400 is preferably performed during surgical planning but can additionally or alternatively be performed during a surgical procedure (e.g., after a first cut on the subject). In variants, a trajectory 20 can be determined: each time a provider changes the trajectory parameter values; when the trajectory 20 intersects a critical structure; for each of a set of target structure—exit region pairs; each time a provider rejects a trajectory 20; when an intersection score falls below a threshold value; when an intersection score meets any other suitable condition, and/or at any other suitable time. In variants, S400 can be iteratively repeated using information generated in step S500 until a trajectory 20 is determined which meets a subjective or objective condition. In an example, a provider can iteratively determine new trajectories, wherein the new trajectories (and associated surgical guides) can be iteratively displayed to the provider. The provider can then select the trajectory 20 (e.g., based on the associated surgical guides). S400 can be performed responsive to one-time provider input, responsive to continuous or intermittent provider input, responsive to feedback from S500, or at any other suitable time.

In variants, S400 can determine the trajectory 20 using any available information, including location of a target 10, a target structure shape, and any other suitable characteristic of target structure, non-target structures, provider-specified “zones” in the skull (e.g., no-go zones, etc.), subject data, previously-tried trajectory parameters, default trajectories, provider structures (e.g., surgical tools) and/or any other suitable data or preferences.

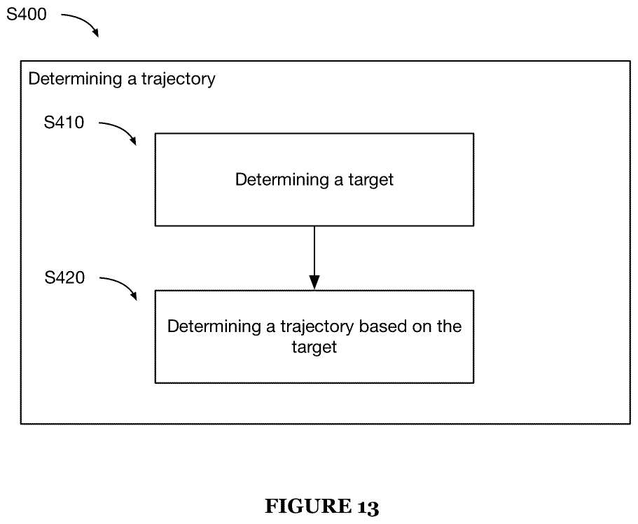

S400 can include determining a target S410, determining a trajectory 20 based on the target S420, and/or any other suitable processes (e.g., example shown in FIG. 13).

Determining a target 410 functions to determine a structure for a cut to terminate at or pass through (e.g., at the location of a tumor, etc.). A target 10 is preferably a target structure (e.g., a tumor) but can additionally or alternatively be or include a point and/or region within the 3D representation 100. Examples of targets 10 include a tumor, clot, implant, organ, virtual region, point within the 3D representation 100, and/or any other structure. S410 can include identifying one or more targets 10. When multiple targets 10 are determined, the trajectory 20 can be determined based on all targets 10, a subset of the targets 10, for each individual target 10, and/or for any other suitable set of targets 10.

The target 10 can be selected by a provider, taken from a data set, automatically determined (e.g., based on rules, predicted using a trained first end region determination model, etc.), or determined through any other suitable method. In a first variant, a provider manually selects a virtual target 10 (and/or a region thereof) existing within the 3D representation 100. In an example of this variant, the provider can manually identify a target 10 in 3D space (e.g., by drawing the target 10, etc.). In a second variant, a target selection module automatically selects a target 10. In this variant, selection can be based on anatomical abnormalities (e.g., determined based on a comparison with a previously-taken scan of the subject, a comparison with another subject of a similar type, based on a model trained to detect anomalies, etc.), based on the type of surgery, and/or any other suitable contextual information. In a first example, an abnormality can be determined from 2D scans (e.g., CT scans) and a corresponding virtual structure 60 in a 3D representation 100 can be identified. In a second example, an abnormality can be determined from the 3D representation 100 directly. However, anatomical abnormalities can otherwise be identified. In a third variant, a virtual structure 60 representing a tagged physical structure (e.g., tagged with a radioactive marker observed in a measurement 300 and/or the 3D representation 100, etc.) can be selected. In this variant, tagging a physical structure can include using radiopaque markers, visible dyes, ultrasound guidance, fluorescent dyes, and/or other suitable types of structure tagging.

Determining a target 410 can additionally or alternatively include determining a first end region (e.g., equivalently referred to herein as “target intersection region”). The first end region can be used to define the first end region of a trajectory 20 or another point relating to trajectory shape. A first end region can be a point, plane, surface, volume, or any other region. A first end region can be within the target 10, determined based on the locations of multiple targets 10, or can be otherwise positioned. A first end region can be a predetermined region relative to the target 10, manually specified, specified based on the angle of the trajectory 20 relative to the surgical entry, or can be any other region. In variants, the first end region can be the center of mass (e.g., assuming constant density) of the structure, the 3D median point, the 3D mode point, the geometric center, a provider-selected region, an automatically selected coordinate (e.g., by a trained model), a plane orthogonal to the trajectory 20 intersecting the target 10, and/or any other suitable region. The first end region can be in a coordinate system relative to: the tumor or other point on the subject, a piece of surgical equipment, the environment, or any other reference point. In an example, the first end region can be automatically determined for a target structure when the target structure is selected by the provider (e.g., the geometric center or center of mass). In a second example, the first end region can be automatically selected to be the center of mass or another point relative to a set of selected structures.

Determining a trajectory based on the target 420 functions to determine a trajectory angle, shape, dimensions, and/or other parameters of the trajectory 20. directionless.

S420 can be performed after target determination, after first end region determination, after second end region determination, or at any other suitable time. S420 can be performed any number of times (e.g., each time a new target 10 is selected, each time a trajectory parameter value is changed, etc.). S420 can be iteratively performed with S500. In an example, the provider modifies the trajectory parameters, visualizes the resulting location and shape of a cut on the surface of the head from S500, and modifies the trajectory parameters again. S420 can additionally or alternatively be performed before, during, or after S300. In an example, S420 can include performing S600 (e.g., determining observation parameters based on a current trajectory, etc.) and S300 (e.g., displaying virtual structures 60 according to the determined observation parameters, etc.).

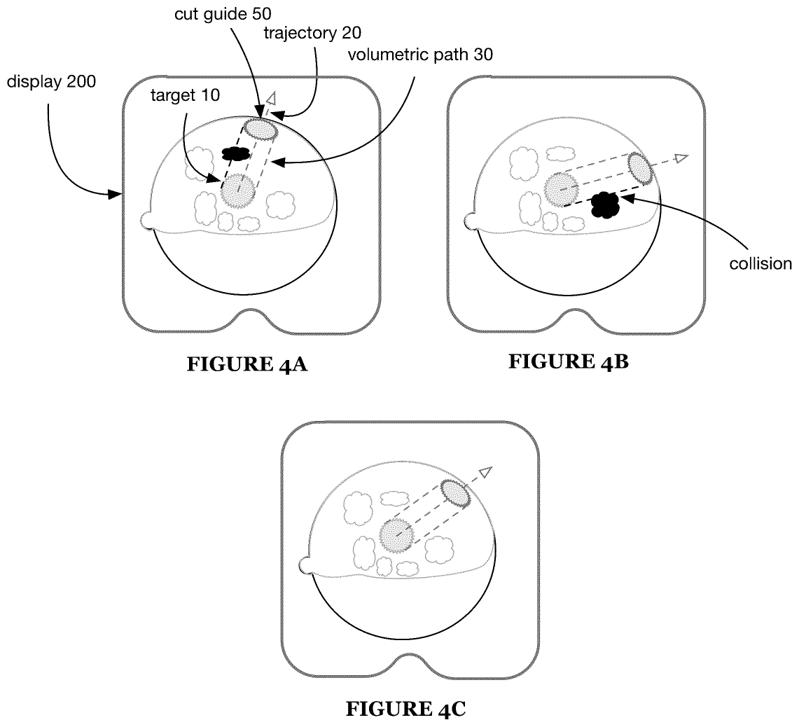

The trajectory parameters can be selected by a provider, taken from a data set, automatically determined, determined through a combination of methods, or determined using any other suitable method. A single trajectory 20 is preferably determined, but alternatively multiple trajectories 20 can be determined. In a variant, a provider determines an initial trajectory 20 and iteratively adjusts the trajectory 20 until a final trajectory 20 is selected (e.g., example shown in FIG. 4A, FIG. 4B, and FIG. 4C). In a second variant, multiple candidate trajectories can determined, and the provider can select a final trajectory 20 or set of trajectories from a set of candidates.

Determination of trajectory parameters can use data determined in other steps. In a first variant, a trajectory's parameters can be automatically edited responsive to: feedback from the provider (e.g., an instruction to change the trajectory 20, etc.), a volumetric path 30 intersecting a surrounding structure (e.g., with a high intersection score, etc.) a trajectory 20 intersecting a surrounding structure, and/or other data. In a second variant, a provider can adjust trajectory parameters based on the visualization of a set of surgical guides (e.g., volumetric path 30, cut guides 50, etc.) determined based on a first trajectory 20 (e.g., determined in S400). In a third variant, a provider can adjust trajectory parameters mid-procedure (S700) after discovering a structure or structure change not present in the 3D representation 100 (e.g., a target tumor grew or shifted, or an organ changed size between the initial 3D scan and the procedure).

In a first variant, trajectory parameters can be predetermined.

In a second variant, trajectory parameters can be determined based on a set of predetermined rules applied to a set of metrics (e.g., an intersection score and/or other metrics), where the rules and metrics can inform which trajectory 20 to select from a group of candidate trajectories, can filter a group of candidate trajectories, or can guide the determination of a trajectory 20 (e.g., through an optimization). Metrics can include the intersection score, whether the trajectory 20 (and/or surgical guide determined using a trajectory 20) intersects or is within a threshold distance of a vital structure, no-go zone, high-brain activity region, surgical structure or tool, and/or any other entity. In an example, a trajectory can be determined using an optimization over the target structure parameters, the adjacent structure metrics, and/or other information (e.g., maximizing target structure access while minimizing an adjacent structure interaction score or penalty score, etc.), or by using another objective function. Metrics can additionally or alternatively include trajectory and/or cut parameters and characteristics (e.g., the angle of the trajectory 20, the distance between the first end region and second end region, etc.). Metrics can be determined in S420 or can be passed to S420 from another step (e.g., preferably S500). In a first example, a trajectory 20 must be orthogonal to the first end region, second end region, target surface, and/or secondary structure surface. In a second example, the trajectory 20 must be the shortest path between the target 10 or first end region and another region (e.g., the surface of the skull). In a third example, the cut created by the trajectory 20 must create the smallest orthographic projection onto the skull (e.g., determined iteratively using different orthographic projections, determined by determining the smallest projection of the target 10 onto the surface of the skull). In a fourth example, the trajectory 20 must be the path inflicting minimal harm, where harm can be a metric relating to a trajectory 20 and/or volumetric path 30 and structures intersecting the trajectory 20 and/or volumetric path 30. In specific examples, harm can be the number of critical anatomical regions (e.g., forbidden regions, critical structures, etc.) intersected, the calculated net magnitude of intersecting a set of regions intersecting a cut or can be any other suitable harm score. In this example, critical anatomical regions can be automatically determined or manually specified.

In a third variant, the trajectory 20 can be determined based on a chosen entry location, where the trajectory 20 can be a vector between the target 10 (e.g., first end region) and the chosen entry location. The chosen entry location can be a point (e.g., a second end region) or region (e.g., based on which a second end region is determined). The chosen entry location can be on a secondary structure, preferably the subject exterior but alternatively another structure. The chosen entry location is preferably defined in virtual space but can alternatively be defined in the real world. The chosen entry location can be representative of the physical entry point into the subject interior or the cut entry point. The chosen entry location can be manually chosen, automatically chosen (e.g., learned, determined according to a set of rules, etc.), and/or determined through any method. The chosen entry location is preferably used as the second end region but the chosen entry location can be otherwise used.

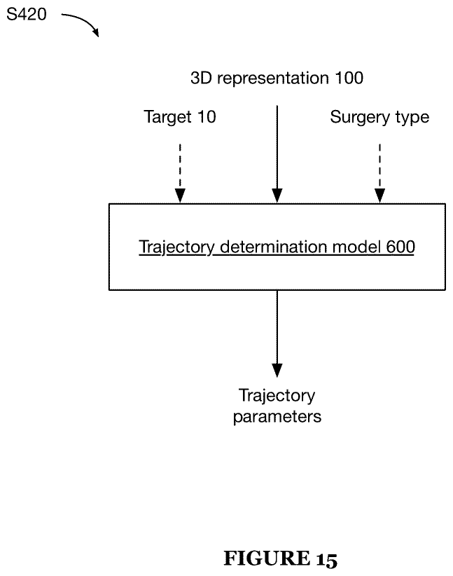

In a fourth variant, trajectory 20 can be determined by a model generated in step S800 (e.g., a trajectory determination model 600; example shown in FIG. 15, etc.), a model generated by a third party, or any other model.

In a fifth variant, trajectory 20 can be manually defined by a provider optionally receiving metric-based feedback (e.g., in S300 and/or S700, based on observation parameters determined in S600, etc.). In this variant, the provider can manually select all or a subset of trajectory parameters via a user interface. In an example, a virtual representation of an initial trajectory 20 can be rendered, and the provider modifies and re-renders the trajectory 20. The virtual representation of the trajectory 20 can be anchored at the target 10 (e.g., the first end region) and/or at the second end region. The provider can adjust the trajectory parameters using a real or virtual “pointer” (e.g., moving the entry-end of the trajectory 20, moving a proximal point of the trajectory 20, etc.), a directional pad (e.g., increment the trajectory angle with each button push, etc.), or any other mechanism to adjust trajectory parameters. In a second example, a provider draws the trajectory 20 in virtual space. In this variant, manually defined trajectories can be further automatically refined.

In this variant, metric feedback transmitted to the provider can further inform the trajectory 20. In an example, an interface presents feedback to a provider as the provider modifies the trajectory 20, where feedback can be a function of a metric or set of metrics. Feedback can be audio, visual, haptic, temperature, pressure, a notification, a suggested trajectory parameter change, a structure color change in the virtual reconstruction, and/or any other type of feedback. Feedback type and/or parameters can be automatically or manually specified. In a first example, when a trajectory 20 intersects a vital structure, a headset (e.g., including the display 200 or separate from the display 200) or control device vibrates. In a second example, when a trajectory 20 comes within a specified distance of a structure labeled “no-go,” a speaker emits a warning sound. Any other suitable feedback type can additionally or alternatively be used.

However, S420 can be otherwise performed.

However, S400 can be otherwise performed.

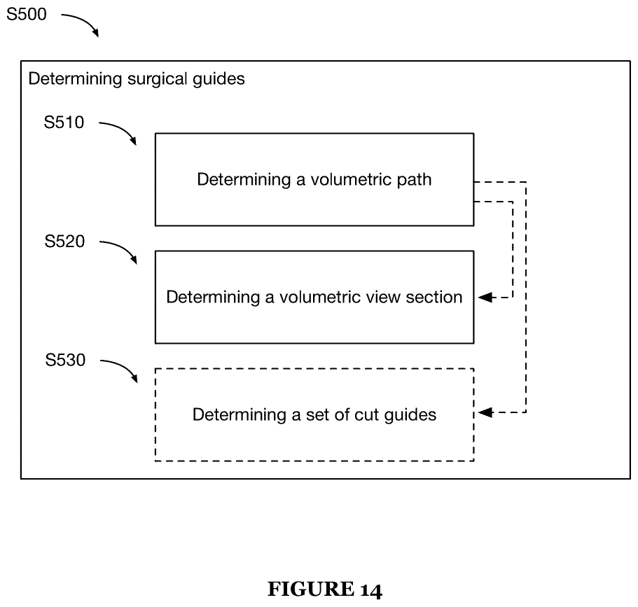

Determining surgical guides S500 functions to determine a set of display information which can assist in surgical planning and during the surgical procedure. S500 is preferably performed iteratively (e.g., as trajectory parameters are re-determined) but can alternatively be performed once or any other suitable number of times. S500 is preferably performed during surgical planning but can additionally or alternatively be performed during a surgical procedure. In an example, S500 can be performed mid-surgery based on measurements 300 captured during the surgery. S500 can include determining a volumetric path S510, determining a volumetric view section S520, determining a set of cut guides S530, and/or any other suitable processes (e.g., example shown in FIG. 14). In an example, determining observation parameters S600 can be determined as part of S500.

Determining a volumetric path S510 functions to determine a representation of a 3D volume through which the target 10 travels. S510 is preferably iteratively performed with S400 but can additionally or alternatively be performed at another suitable time. The volumetric path 30 is preferably based on a path profile and the trajectory 20 but can alternatively be performed based on the target 10 and a cut and/or cut guide 50 on the secondary structure or based on any other suitable combination of surgical planning information. The path profile and/or trajectory can be manually determined, automatically determined (e.g., based on the target structure dimensions, based on tool dimensions, etc.), and/or otherwise determined.

The volumetric path 30 is preferably generated by projecting (e.g., extruding) the path profile between the first end region and the second end region, but the volumetric path 30 can be alternatively determined. In a first variant, the volumetric path 30 can be generated by projecting the path profile onto a second end region (e.g., a secondary structure surface) along the trajectory 20. In a second variant, the volumetric path 30 can be generated by moving a virtual instance of the target 10 along a path (e.g., the trajectory 20 or a non-trajectory path) until a provider-specified height can be reached and/or the virtual target instance intersects the secondary structure surface; then generating a 3D path between the target 10 a virtual target instance. In a third variant, the volumetric path 30 can be determined by lofting the target cross-section or silhouette to a cut profile (e.g., a 3D or 2D path on the surface of the subject). In this variant, “lofting” can refer to connecting (e.g., blending, interpolating, etc.) multiple profiles to generate a 3D shape. In this variant, the cut profile can be a cut guide 50 determined in S530, can be manually traced by a provider, and/or can otherwise be determined. The path profile of the volumetric path 30 preferably does not rotate, change size, and/or change shape along the trajectory 20 but can alternatively rotate, change size, and/or change shape along the trajectory 20. However, S510 can be otherwise performed.

Determining a volumetric view section S520 functions to determine a representation of a 3D volume which facilitates viewing of the target 10 during the procedure and/or during procedure planning. S520 is preferably performed after S510 but can additionally or alternatively be performed at another suitable time. S520 is preferably performed based on a volumetric path 30 (e.g., determined in S510) but can additionally or alternatively be based on a trajectory 20.

In a first variant, the volumetric view section 40 can be determined by extruding a view section profile (e.g., using any of the methods described in variants of volumetric path determination above, using the view section profile in place of the path profile, etc.). In this variant, the view section profile is preferably the same shape as the path profile but can alternatively have a shape which is based on the path profile or determined independently of the path profile. In examples, the view section profile can be determined by adding a margin (e.g., 2D margin) to the path profile or trajectory, by smoothing the path profile, by determining a similar shape to the path profile, and/or by performing any suitable shape determination method. In this variant, the view section profile is preferably larger than the path profile but can alternatively be the same size as the path profile.

In a second variant, the volumetric view section 40 can be determined by adding a volumetric outer margin to the volumetric path 30 or trajectory. The margin magnitude (e.g., distance between the path profile boundary and the volumetric view section boundary can be manually determined, automatically-determined, and/or otherwise determined. In examples, the margin magnitude can be 0.1 inches, 0.5 inches, 1 inch, 2 inches, within an open or closed range bounded by any of the aforementioned values, and/or within any other suitable range. The margin magnitude preferably increases with distance from the first end region and/or target 10 but alternatively can be constant or otherwise dynamic along the trajectory 20.

In a third variant, the volumetric view section 40 can be determined by adding a volumetric outer margin to the trajectory 20. In this variant, the margin magnitude can refer to the distance between the trajectory 20 and the boundary of the volumetric view section 40, and can have any of, all of, or none of the characteristics of the margin magnitude in the previous variant (e.g., the second variant).

In a fourth variant, the volumetric view section 40 can be any a union of any of the aforementioned variants, the target 10, and optionally a volumetric margin around the target 10. However, the volumetric view section 40 can be performed through any other suitable means.

However, determining a volumetric view section S520 can be otherwise performed.

Optionally determining a set of cut guides S530 functions to determine representations of cuts on a cut surface. S520 can also function to enable construction of a repair apparatus (e.g., a replacement bone flap).

The cut surface can be a surface of a subject (e.g., a surface of an external or internal structure, etc.), a surface of a virtual representation of a subject structure (e.g., a virtual structure 60, etc.), and/or any other suitable surface. Examples of structures on which a cut surface can lie include an external structure, internal structure, target structure, secondary structure, surrounding structure, and/or any other suitable structure.

S530 can be performed iteratively with and/or after S400, S510, S520, and/or at any other time.

A cut guide 50 can include only the margin (e.g., the perimeter of the tumor) or can include the full interior shape of the cut. A cut guide 50 is preferably a representation of a physical cut on a physical secondary structure (e.g., a subject's exterior, a secondary internal anatomy, etc.), but the cut guide 50 can additionally or alternatively be a representation of a cut on any other suitable structure.

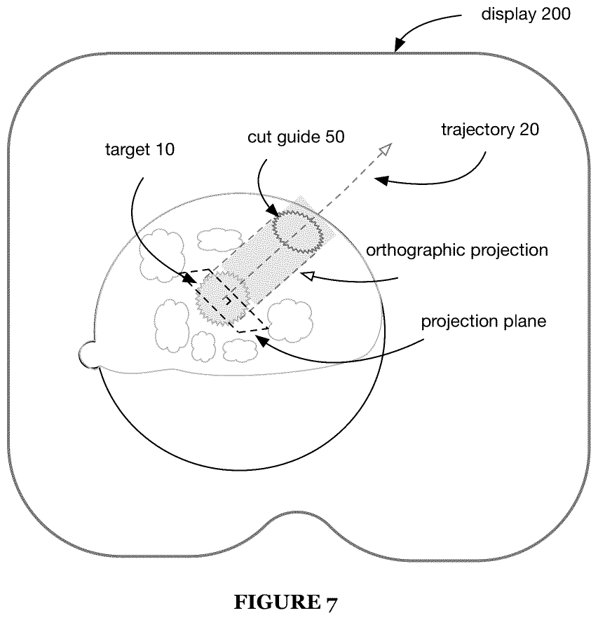

In a first variant, a cut guide 50 can be determined by projecting the target 10 onto a plane orthogonal to the trajectory 20, then projecting the target projection from the plane onto a cut surface (e.g., a secondary structure, a virtual structure 60 representing a secondary structure in the 3D representation 100, etc.) along the same trajectory 20 (e.g., example shown in FIG. 7) and/or a different trajectory 20.

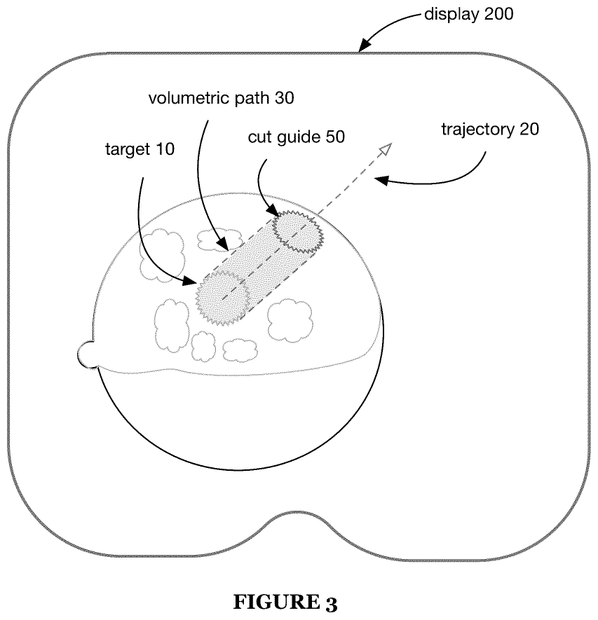

In a second variant, a cut guide 50 can be determined by projecting the target 10 directly onto a secondary structure surface (e.g., orthogonal to the trajectory 20) (e.g., example shown in FIG. 3). The projection is preferably performed in virtual space, wherein the target structure projection can be projected onto a virtual representation of the secondary structure. The virtual representation of the secondary structure can be determined as part of the 3D representation 100 (e.g., from the first set of measurements 300), be part of the subject representation, and/or be otherwise determined.

In a third variant, a cut guide 50 can be determined by generating a cross section of a target 10 and projecting the cross section onto the cut surface along the trajectory 20. In this variant, the cross section of the target 10 can be: the cross section orthogonal to the trajectory 20 at the first end region, a minimum cross section, a maximum cross section, or any other suitable cross section.

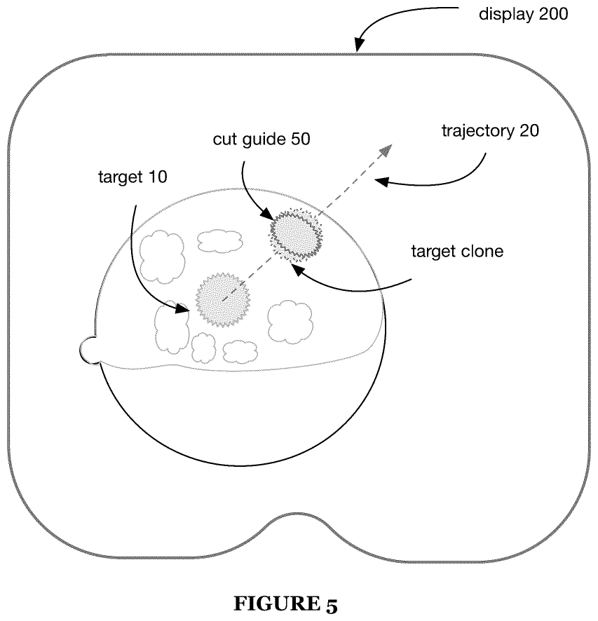

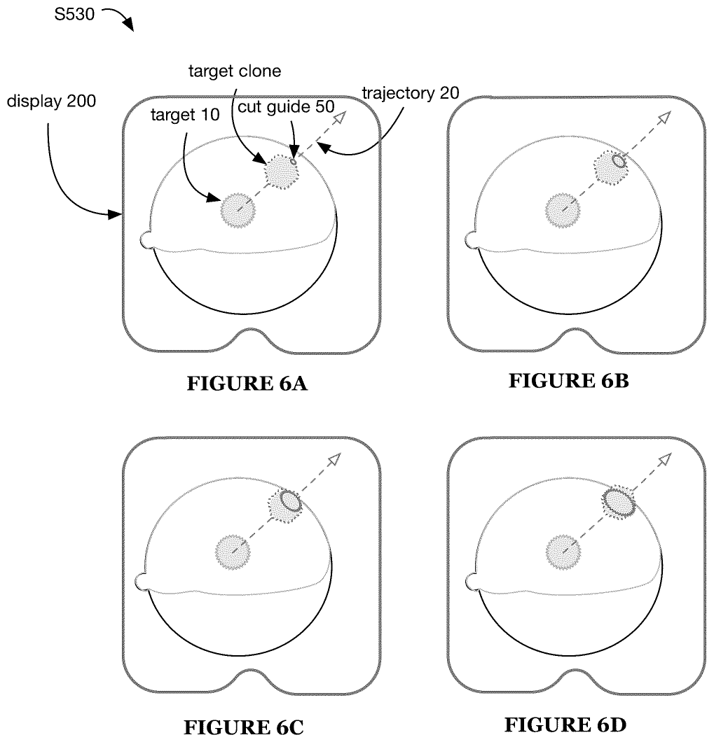

In a fourth variant, a cut guide 50 can be determined by moving a virtual clone of the target 10 along the trajectory 20 until a target position is achieved (e.g., example shown in FIG. 5). The target position can be: a provider-specified height, a second end region, a position where the target clone instance intersects the cut surface (e.g., example shown in FIG. 6A, FIG. 6B, FIG. 6C, and FIG. 6D), a position where more than a threshold proportion of the target clone extends beyond the cut surface, and/or be otherwise defined. The cut guide 50 can be defined by the intersection of the cut surface (e.g., external surface, internal surface, secondary structure thickness, etc.) and the target clone. The intersection is preferably determined in virtual space, but can alternatively be determined in real space, augmented reality space, and/or in any other suitable space. In a first embodiment, the intersection can be determined in virtual space, wherein both the target clone and the cut surface can be virtually represented. In this embodiment, only a virtual representation of the target clone can be shown to the provider; only virtual representations of the target clone and intersection region can be shown to the provider; virtual representations of the target clone, the intersection region, and the cut guide 50 surface can be shown to the provider; and/or any other suitable virtual structure 60 can be displayed. In a second embodiment, the cut guide 50 can be defined by the intersection of the real-world subject and the virtual target clone. Alternatively, a provider (e.g., surgeon) can trace a perceived intersection (e.g., onto the physical surface of the subject) to define the cut guide 50.

In a fifth variant, a cut guide 50 can be determined by moving a target clone (e.g., a virtual structure) along the trajectory 20 until the target structure clone intersects a plane tangent or is proximal to the structure surface, then taking the cross section of the target clone at the plane and using the cross section as the margin of the cut guide 50. In an example, characteristics of a target clone cannot be changed, but alternatively characteristics can be changed (e.g., rotation, size of the target clone).

In a sixth variant, any combination of any element of the aforementioned variants can be used to determine the cut guide 50.

However, S530 can be otherwise performed.

However, S500 can be otherwise performed.

Determining observation parameters S600 functions to determine how an observed element (e.g., surgical guides and/or virtual structures 60, etc.) is shown on the display 200 (e.g., in S300 and/or S700, etc.). S600 can be performed iteratively with S400, iteratively with S500, before a next iteration of S300 and/or S700, and/or at another suitable time. In a first example, S600 facilitates trajectory determination in S400 by dynamically highlighting intersecting structures with a trajectory and/or surgical guide. In a second illustrative example, S600 facilitates the procedure in S700 by dynamically highlighting intersecting structures with a surgical tool.

An observed element can be a surgical guide (e.g., a cut guide 50, a volumetric path 30, a volumetric view section 40), virtual structures 60 (e.g., the target structures, surrounding structures, internal structures, external structures, subject structures, provider structures, etc.) and/or a portion thereof, a trajectory 20, and/or any other suitable information or portion thereof.

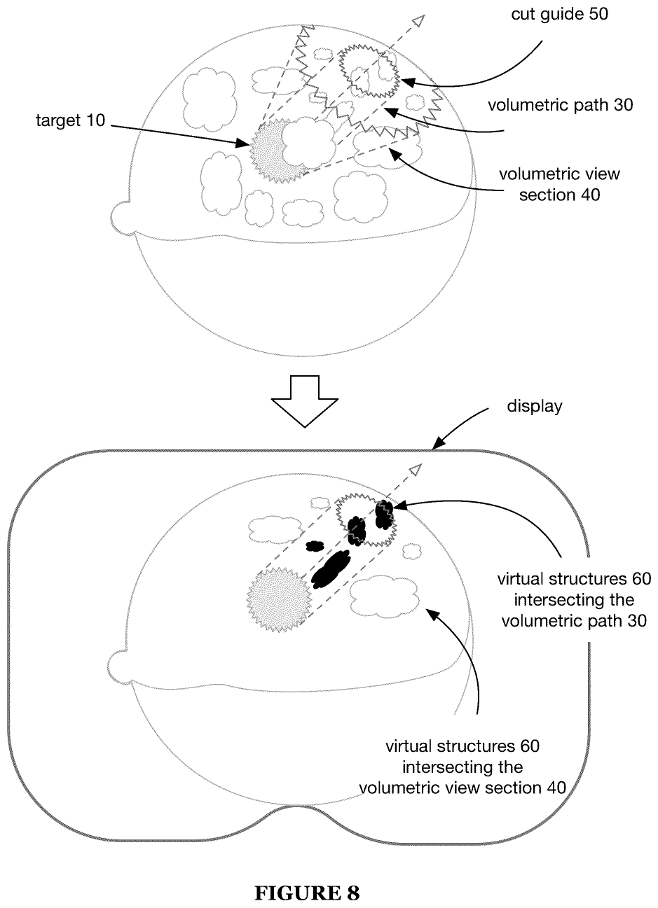

Observation parameters can include, for each observed element and/or portion thereof: transparency, presence, pose, color and texture, dimensions (e.g., shape, size, ratio relative to the target structure, etc.), brightness, haptic feedback associated with an intersecting reference element (e.g., a provider structure intersecting a critical surrounding structure, etc.), an observed element label (e.g., labeled with structure type, intersection score, etc.), and any other parameter relating to the visibility of virtual structures 60 within the 3D representation 100. Observation parameters can be manually determined, determined based on a set of display rules or conditions, or can be otherwise determined. Examples of display rules include “only show structures which intersect a volumetric path and do not obstruct a view of the target,” (e.g., example shown in FIG. 8) “only show structures with a specific label,” “show structures within N mm of the trajectory,” and “only show skeletal structures,” but other display rules can additionally or alternatively be used.

Observation parameters can be determined automatically, determined manually, determined according to a set of presets, selected from a set of recommended parameters, changed automatically responsive to other parameters being changed, input by a provider, and/or be otherwise determined. The provider input (e.g., provider-provided input, etc.) can be received from virtual buttons within the display 200 (e.g., AR, VR, XR, etc.), a physical interface (e.g., keyboard, mouse, button, etc.), voice commands, eye gaze tracking or other body language commands, and/or other provider input systems. Observation parameters can be set, reset, and/or adjusted at any time.

Observation parameters can be based on an observed element's intersection with a reference element, based on an observed element's proximity to a reference element, based on an observed element's type (e.g., structure type), based on a reference element's type, based on a surgery type, based on a current action type, based on an intersection score of the trajectory 20, based on an observed element's criticality score, and/or based on other suitable information. The reference element can be a target 10, a subject structure (e.g., target structure, surrounding structure, etc.), a provider structure (e.g., a surgical tool), a surgical guide (e.g., a volumetric path 30, volumetric view section 40, a cut guide 50, etc.), and/or any other suitable element and/or virtual representation thereof.

In a first embodiment, observation parameters for an observed element can be based on the observed element's intersection with a reference element.

In a first variant of this embodiment, an observation parameter for a whole observed element (e.g., a whole virtual structure 60, etc.) can be based on whether the observed element intersects a reference element. In a first example, when an observed element (e.g., a virtual structure 60) partially overlaps a reference element (e.g., a volumetric view section 40), the observed element can be visible and invisible otherwise. Alternatively, the visibility of the observed element can be reversed (e.g., visible when non-overlapping, etc.). In a second example, when a reference element (e.g., a virtual representation of a tool tip, etc.) touches an observed element (e.g., a virtual structure 60, etc.), the observation parameters for the observed element change (e.g., the virtual structure 60 changes color, lights up, becomes visible, etc.).

In a second variant of this embodiment, an observation parameter for a region of an observed element can be based on whether the region intersects a reference element. In an example, when an observed element partially overlaps a surgical guide, the overlapping region can be invisible, and the non-overlapping section can be visible. Alternatively, the visibility of the observed element can be reversed (e.g., visible when non-overlapping, etc.).

In a third variant of this embodiment, an observation parameter for a slice of an observed element can be based on whether and/or how the slice intersects the boundary of a reference element. In an example, when an observed element partially overlaps a surgical guide, a slice of the observed element can be generated and can be visible at the boundary.

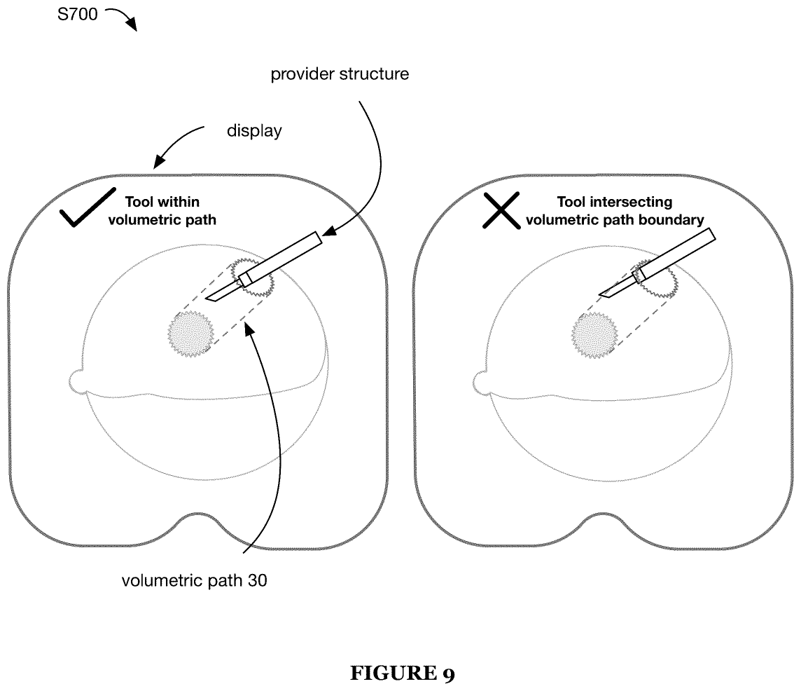

However, the observation parameters for an observed element can otherwise be based on the observed element's intersection with a reference element (e.g., example shown in FIG. 9).

In a second embodiment, observation parameters for an observed element can be based on a proximity to a reference element. In an example, an observation parameter changes based on a proximity of a reference structure (e.g., a surgical tool) to the boundary and/or center of the observed element.

In a third embodiment, observation parameters for an observed element can be based on an intersection score (e.g., referred to equivalently herein as a “trajectory score”), criticality scores, and/or other metrics. In a first example, observation parameters for an observed element can be based on a criticality score specific to the observed element. In a second example, observation parameters for an observed element can be based on an intersection score specific to a current trajectory 20. In a third example, observation parameters can be based on a weighted criticality of an intersected structure with a surgical guide, weighted based on the intersection volume relative to the overall volume of the intersected structure (e.g., how much a volumetric path 30 overlaps a virtual structure 60, etc.). In this embodiment, the observed element can be a surgical guide, a virtual structure 60, and/or any other suitable observed element type.

In a fourth embodiment, observation parameters for an observed element can be based on eye tracking. In an example, the observation parameter depends on whether a provider is looking at or near the observed element.

However, S600 can otherwise be performed.

Facilitating the procedure S700 functions to use the cut geometry to aid a provider performing a procedure on a subject represented in the 3D representation 100. Facilitating the procedure S700 can occur contemporaneously with S400 and/or S500, and/or at any time. Facilitating the procedure preferably includes S300 and/or the processes described in S300 but can alternatively be independent of S300 and the processes described therein.

S700 can include displaying the 3D representation 100, a subset of virtual structures 60 within the 3D representation 100 (e.g., structures relevant to the present procedure, etc.), a measurement 300, a representation of a surgical tool, a representation of provider structures, and/or any other suitable elements. S700 can optionally include tracking features in the measurements (e.g., fiducials, keypoints, etc.) and aligning the 3D representation based on the tracked features and the registration. S700 can optionally additionally or alternatively include displaying cross-sections of any of the aforementioned elements.

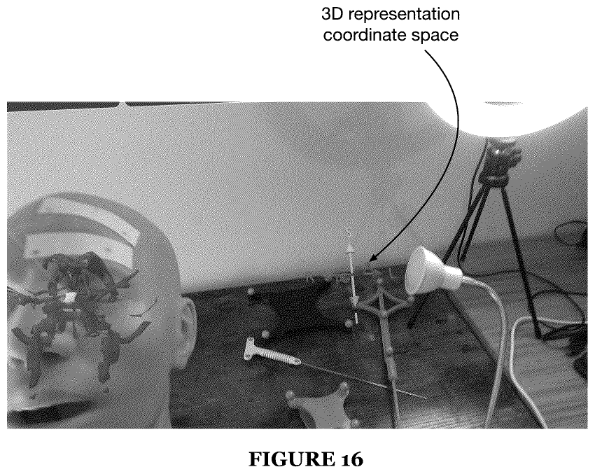

S700 can include displaying the elements from the perspective of a sensor 400 which captured a measurement 300 and/or from the pose of a virtual sensor 1000 (e.g., in a virtual measurement). In a virtual sensor variant, the pose of the virtual sensor 1000 can be defined in world space, defined in a 3D representation coordinate space (e.g., example shown in FIG. 16), defined in a subject coordinate space, defined in a provider coordinate space and/or defined in any other suitable coordinate system. The pose of the virtual sensor can be defined relative to a subject and/or structure thereof (e.g., a target 10, a surrounding structure, etc.); a provider and/or structure thereof (e.g., a surgical tool); and/or any other suitable reference point. In the virtual sensor variant, the virtual sensor 1000 is preferably a perspective camera but can alternatively be an orthographic camera (e.g., wherein a projection of a target cross-section, boundaries thereof, and/or other derivative visualizations can be displayed). S700 can additionally or alternatively include communicating non-visual information (e.g., sounds, vibrations, audio instructions, etc.). In examples, the pose of the virtual sensor 1000 can be a sagittal right pose, a sagittal left pose, a coronal anterior pose, a coronal posterior pose, an axial superior pose, an axial inferior pose, and/or any other suitable pose (e.g., relative to the reference frame). Virtual measurements generated by the virtual sensor 1000 can depict the same layers or different layers as are visible through a sensor 400. For example, the virtual sensor can depict only a certain anatomy type (e.g., a target 10, a type of surrounding structure, etc.), a subset of surgical guides, and/or any other suitable information. In example, the virtual sensor can generate a virtual measurement depicting surgical guides (e.g., cut guide 50, volumetric path 30, volumetric view section 40, etc.), labels, a target 10, surrounding structures, subject structures, provider structures, and/or any other suitable information.

In a variant, facilitating the procedure includes representing virtual structures 60, surgical guides, a trajectory 20, and/or other suitable system components on a 3D model depicted in a 2D display 200 (e.g., a screen displaying a subject representation and/or the cut guide 50).

In a second variant, facilitating the procedure can include representing virtual structures 60, a 3D representation 100, surgical guides, and/or a trajectory 20 on a subject representation in a 3D display 200 (e.g., an AR/XR/VR headset); example shown in FIG. 3.

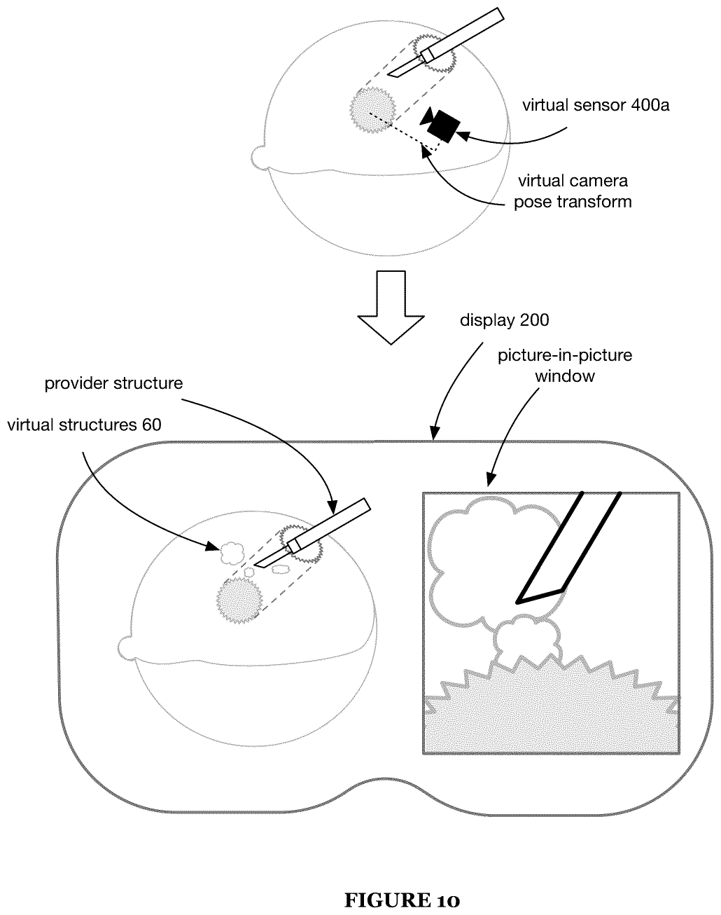

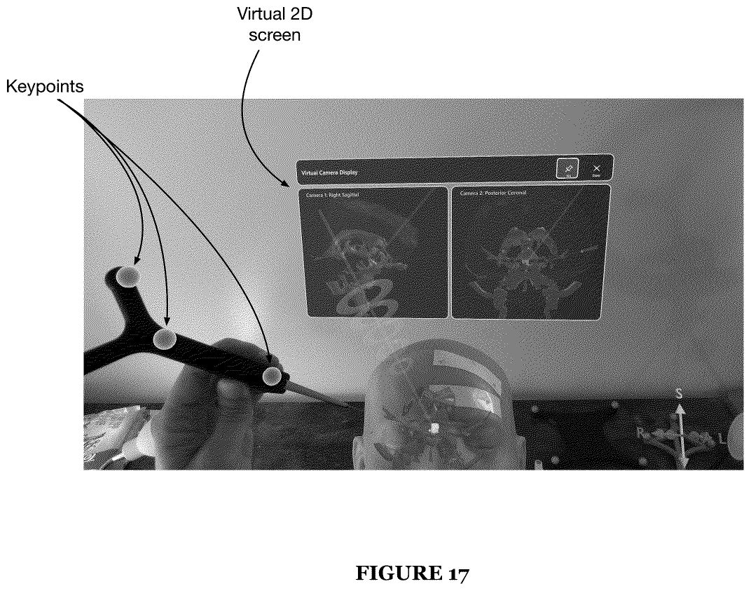

In a third variant, facilitating the procedure can include representing virtual structures 60, surgical guides, and/or a trajectory 20 on a virtual 2D screen within the display 200 (e.g., example shown in FIG. 10). In a first example, S700 includes representing virtual structures 60, surgical guides, and/or a trajectory 20 on a picture-in-picture overlay on the display 200. In a second example, S700 includes representing virtual structures 60, surgical guides, and/or a trajectory 20 on a picture-in-scene representation for a 3D display 200 (e.g., a floating virtual screen within an AR/VR/XR representation; example shown in FIG. 17). In examples of this variant, a single virtual screen can be displayed, or multiple virtual screens can be displayed. In examples with multiple virtual screens, the virtual 2D screens can depict information (e.g., virtual measurements) generated using virtual cameras 1000 with the same pose or different poses as other virtual 2D screens. The virtual 2D screens can depict different information (e.g., information captured using different observation parameters, different anatomical structures, etc.) and/or the same information as other virtual 2D screens and/or a main display (e.g., a main subject representation of the patient). However, a virtual 2D screen can otherwise be displayed.

In a fourth variant, facilitating the procedure can include using a light projector to project virtual structures 60, surgical guides, and/or a trajectory 20 onto a subject's physical body. In an example, the cut guides 50 can be projected onto subject structures in the position and orientation of the cut guide 50.

In a fifth variant, facilitating the procedure includes providing feedback (e.g., notifications, haptic feedback, etc.) to a surgeon if provider structures or virtual representations thereof stray near or outside the bounds of a target 10, planned trajectory 20, and/or surgical guide (e.g., cut guide 50, volumetric path 30, etc.).

In a sixth variant, facilitating the procedure includes automatically generating a surgery plan for use by a surgical team in preparing for surgery.

In a seventh variant, facilitating the procedure includes exporting a 3D shape file of the surgical guides, trajectory 20, target 10, virtual structures 60, and/or any other suitable information.

However, S700 can be otherwise performed.

Optionally determining a set of models S800 functions to train a set of models to determine trajectory parameters and/or any other suitable information to facilitate a procedure. Examples of models that can be determined can include a trajectory determination model 600 and/or any other model. In an example, the trajectory determination model 600 is preferably determined before S100 and updated after S400 and/or S700. Alternatively, the trajectory determination model 600 can be determined and/or updated at any other suitable time.

Models can determine: target structures, trajectories, cut guides 50, and/or other parameters based on: a set of 3D structures, information about a subject's body, information about a target 10, information about available surgical tools and methods, time constraints, procedure type, target structure parameters, secondary structure parameters, trajectory constraints, cut constraints, a given trajectory 20, and/or any other type of information. Models can include: neural networks, transformers, and/or other models.

The models can be trained on (e.g., training targets can include): prior selected trajectories (e.g., trajectories selected during previous surgeries, previous iterations of the S400 step, and/or trajectories determined through any other method).

Training data can be generated by providers determining trajectories and/or cut guides 50 on synthetic 3D representations, modified 3D reconstructions, 3D representations of subjects undergoing or not undergoing surgery, and/or any other 3D model or combination of 3D models.

S800 can be performed responsive to any suitable condition. In a first variant, a model can be updated (e.g., refined, etc.) when new procedure data becomes available. In a second variant, a model can be wholly retrained on data relating to a new set of procedures. In an example, a model can be trained and/or refined on subject-specific data and/or on data relating to a present subject (e.g., data relating to subjects with the same condition, subjects of the same sex, and/or subjects grouped on any other suitable basis).

Provider and subject behaviors recorded during S400, S500, and S700 can be recorded and used as training data for the trajectory determination model. In a variant which uses an intersection of a model of a target 10 elevated along the trajectory 20, the chosen height of the target clone can be recorded and stored for use as training data. Additionally or alternatively, non-chosen heights can be recorded and stored for use as training data. In any variant in which a trajectory 20 is determined, the chosen trajectory angle and trajectory parameters can be recorded and stored for use as training data. Chosen cut parameters, observation parameters, surgery outcomes, surgery costs, and/or any other data relating to the procedure can additionally or alternatively be stored and used as training data for a model.

The method can be performed at any time. All or portions of the method can be performed in real time (e.g., responsive to a request), iteratively, concurrently, asynchronously, periodically, and/or at any other suitable time. All or portions of the method can be performed automatically, manually, semi-automatically, and/or otherwise performed.

All references cited herein are incorporated by reference in their entirety, except to the extent that the incorporated material is inconsistent with the express disclosure herein, in which case the language in this disclosure controls.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system 500, cause the processing system 500 to perform the method(s) discussed herein. The instructions can be executed by computer-executable components integrated with the computer-readable medium and/or processing system 500. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system 500 (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method for displaying surgical information, comprising:
determining a 3D model of a patient generated based on a set of computed tomography (CT) scans of the patient, wherein the 3D model comprises an internal virtual structure;
identifying a plurality of mapping points in the 3D model;
receiving a measurement of the patient captured using a sensor;
registering the 3D model with the measurement by matching a set of features within the measurement with mapping points from the plurality of mapping points;
determining a trajectory which intersects a first endpoint on the internal virtual structure and intersects a second endpoint proximal to a surface of the patient;
generating a volumetric view section based on the internal virtual structure and the trajectory, wherein:
a dimension of the volumetric view section is based on a dimension of the internal virtual structure; and
the volumetric view section spans the first endpoint and the second endpoint;
selecting a set of virtual structures from the 3D model which intersect the volumetric view section; and
in a display, overlaying a representation of the selected set of virtual structures and a representation of the trajectory onto the measurement based on the 3D model registration.

2. The method of claim 1, wherein the volumetric view section comprises a dimension at least as large as a maximum dimension of the internal virtual structure.

3. The method of claim 1, further comprising generating a volumetric path along the trajectory, wherein a cross-sectional shape of the volumetric path is based on the shape of the internal virtual structure.

4. The method of claim 3, further comprising visually highlighting virtual structures from the set of virtual structures that intersect the volumetric path.

5. The method of claim 3, wherein determining the trajectory comprises adjusting the trajectory based on a minimization of penalties associated with virtual structures intersecting the volumetric path generated along the trajectory.

6. The method of claim 1, wherein generating the volumetric view section comprises projecting a cross section of the internal virtual structure along the trajectory to the second endpoint.

7. The method of claim 6, wherein the cross section has an irregular boundary.

8. The method of claim 1, wherein the volumetric view section comprises a tapered 3D shape increasing in diameter between the first endpoint and the second endpoint.

9. The method of claim 1, further comprising:
determining a modified trajectory;
selecting a new set of virtual structures from the 3D model based on the modified trajectory; and
in the extended reality headset, overlaying a representation of the new set of virtual structures based on the 3D model registration.

10. The method of claim 9, further comprising:
determining a 3D position of a pointer detected in a new measurement; and
determining a new second endpoint based on the 3D position of the pointer, wherein the modified trajectory intersects the new second endpoint.

11. The method of claim 1, wherein the display is within an extended reality (XR) headset.

12. A method, comprising:
receiving a 3D model of a patient generated using a set of cross-sectional scans of the patient, wherein the 3D model comprises a target structure;
identifying a plurality of mapping points in the 3D model; and
iteratively:
determining a measurement of the patient using a sensor;
registering the 3D model with the measurement by matching a set of features within the measurement with the plurality of mapping points; and
in an extended reality headset, overlaying, onto the measurement based on the registration:
a volumetric path, determined based on the target structure, that extends from the target structure to the second point along a trajectory; and
a set of virtual structures from the 3D model, wherein virtual structure inclusion in the set of virtual structures is based on an intersection of the respective virtual structure with a volumetric view section surrounding the volumetric path.

13. The method of claim 12, wherein an appearance of each virtual structure in the set of virtual structures is determined based on a pose of the respective virtual structure relative to the volumetric path.

14. The method of claim 12, wherein 3D model does not comprise the set of cross-sectional scans.

15. The method of claim 12, further comprising, during a surgical procedure, iteratively:
capturing a surgical procedure measurement;
registering the 3D model with the surgical procedure measurement; and
determining a position of a surgical tool depicted within the surgical procedure measurement relative to the 3D model.

16. The method of claim 15, further comprising, responsive to the surgical tool contacting a boundary of the volumetric path, notifying a surgical provider via the display.

17. The method of claim 15, further comprising:
determining a virtual camera pose, wherein the virtual camera pose is defined relative to the 3D model;
generating an image from a perspective of the virtual camera pose, the image depicting:
a subset of virtual structures of the 3D model; and
a virtual representation of the surgical tool at the position of the surgical tool; and
in the extended reality headset, displaying the image.

18. The method of claim 17, wherein displaying the image comprises displaying the image as a picture-in-picture overlay.

19. The method of claim 12, wherein overlaying the set of virtual structures from the 3D model further comprises overlaying a set of 3D cut paths on surfaces of the set of virtual structures, wherein the set of 3D cut paths is determined based on boundaries of the target structure.

20. The method of claim 19, wherein the set of 3D cut paths comprises cut paths on virtual structures representing internal anatomy of the patient.
